(19) United States
(12) Patent Application Publication
(10) Pub. No.: US 2024/0343775 A1
(43) Pub. Date: Oct. 17, 2024

(54) CHIMERIC ANTIGEN RECEPTORS (CAR) FOR B CELL MALIGNANCIES

(71) Applicant: CELLOGEN THERAPEUTICS PVT. LTD., Delhi (IN)

(72) Inventor: Gaurav KHARYA, Delhi (IN)

(73) Assignee: CELLOGEN THERAPEUTICS PVT. LTD., Delhi (IN)

(21) Appl. No.: 18/683,141

(22) PCT Filed: Aug. 12, 2022
(86) PCT No.: PCT/IN2022/050727

§ 371 Date: Feb. 12, 2024

(30) Foreign Application Priority Data
Aug. 12, 2021 (IN) 202111035166

(51) Int. Cl. C07K 14/725; A61K 38/00; A61P 35/02; C07K 16/28; C12N 15/86

(52) U.S. Cl. CPC C07K 14/7051; A61P 35/02; C07K 16/2803; C07K 16/2887; C12N 15/86; A61K 38/00; C07K 2317/31; C07K 2317/622; C07K 2319/02; C07K 2319/03; C12N 2740/15043

(57) ABSTRACT

The present invention relates to chimeric antigen receptors (CAR) comprising of two or more binding domains that binds CD19, CD20 or CD22 and at least two or more intracellular co-stimulatory signaling domains selected from the group consisting of CD28, 41BB, OX40 and ICOS. The CAR may further comprise of a hinge or spacer region, a transmembrane region, an intracellular signaling domain and a linker region. The CAR and compositions thereof of the invention is used in the treatment of B-cell malignancies.

### FIELD OF INVENTION

The present invention pertains to the field of immunology. More particularly, the invention relates to third-generation and fourth generation chimeric antigen receptors (CARs) for the treatment of cancers.

### BACKGROUND OF THE INVENTION

Cancer is a devastating disease that takes the lives of millions of people every year. Due to the heterogeneity in the underlying causes of cancer, traditional treatment modalities with standardized drugs produce variable treatment outcome and often result in treatment failure.

This inherent variability of underlying causes of cancer has given rise to the development of newer therapeutic modalities.

One approach in cancer treatment modalities which has gained huge popularity is CAR-T cell therapy. CAR-T cells are immune cells that are artificially engineered to express a particular chimeric antigen receptor (CAR), which have shown great potential in killing cancer cells. Chimeric antigen receptors (CARs) are artificially produced recombinant receptors having both antigen-binding and T-cell activating potential.

CARs are composed of an extracellular recognition domain sourced from monoclonal antibodies (mAbs) and an intracellular signaling domain including CD3- and costimulatory domains for triggering endogenous signaling pathways of T-cells.

The recognition domain of the CAR can target specific antigen molecules expressed on the surface of cancer cells, thereby triggering unique responses including activation and killing activity of T-cells mediated by the signaling domain.

Till date, several CAR-T cell therapies have been developed. In the first generation of CAR-T therapies, the intracellular domain consists of CD3 ζ-chain or FcεRIγ, and it is the primary transmitter of signals from endogenous T cell receptors (TCR). However, these CAR-T cell therapies are not able to produce enough interleukin-2 (IL-2) to kill tumor cells. Most of the studies conducted with first-generation CAR-T cells could not achieve the desired effects because of inadequate proliferation, a short life span in vivo and inadequate secreted cytokines.

In the second generation of CAR-T cell therapies, additional signaling domains were added. The intracellular domain consisted of T-cell receptors (TCR), cytokine receptors and costimulatory receptors. Presently, some clinically approved CAR design relies on second-generation CARs which are directed against the CD19 antigen, a cell surface receptor more abundantly expressed in malignant B cells. These CD19 directed lentiviral CAR constructs comprise of anti-CD19 scFv derived from a murine FMC63 monoclonal antibody and only one costimulatory domain. Such 2^{nd} generation CARs have shown promising results in patients with refractory and relapsed tumours, where other treatment methods like chemotherapy, radiation therapy and surgery have failed.

Despite this notable efficacy, a large proportion of patients who received a second-generation CAR T-cell infusion experience, progressive disease (PD), owing to a lack of CAR T-cell persistence and tumour cell resistance caused by antigen escape/loss or reduced antigen expression below the threshold required for efficient CAR T-cell activity. Thus, there is inadequate proliferation, a short life span in vivo, and chances of relapse or recurrence of cancer using second-generation CAR T-cell treatment. The limitations with exisiting second generation CAR designs have been described in some recent studies (Abate-Daga et al, 2019; Roselli et al, 2021; Guedan et al, 2019; Gumber & Wang, 2022; Shah et al, 2021).

Another limitation of the exisiting CAR-T cell therapy is the tumor escape mechanism or tumor resistance to single antigen targeting CAR constructs. This tumor resistance arises due to the loss of the tumor specific antigen such as CD19 against which most of the CAR-T cell therapies have been developed. The tumor resistance is multifactorial which includes transcriptional suppression of the CD19 antigen or genetic modifications of the CD19 gene, which leads to complete loss of the CD19 antigen. These challenges have been addressed in some recent scientific reviews (Sterner and Sterner; 2021; Stoiber eta al; 2019; Rafiq et al; 2019).

Due to the above disadvantages in the prior art, there is a need and scope for development of improved CAR-T cell therapies for targeting cancers. Thereby, implying that many opportunities still exist to improve outcomes and address areas of high unmet need in cancers such as B-cell malignancies.

The inventors have contemplated an approach for engineering chimeric antigen receptors to produce third-generation bispecific CAR-T cell therapies with two scFv domains in tandem along with two different co-stimulatory signaling units in the intracellular regions and a signaling domain based on CD36. Similarly, 4^{th} generation bi-specific CAR T cells are based on two scFv domains in tandem along with three co-stimulatory signaling units in the intracellular regions and a signaling domain based on CD3ζ. The greatest advantage of the third and 4^{th} generation CARs is that it can effectuate high levels of cytokine secretion and considerably increase proliferation speed and survival rate of engrafted T-cells and decreases the chances of relapse or recurrence of cancer and tumor heterogenicity.

### BRIEF DESCRIPTION OF FIGURES

The accompanying figures illustrate some of the embodiments of the present invention and, together with the descriptions, serve to explain the invention. These figures have been provided by way of illustration and not by way of limitation.

FIG. 1 provides schematic diagram of CAR construct comprising CD20+CD19 binding domains with OX40 and 41BB co-stimulatary domains.

FIG. 2 provides schematic diagram of CAR construct comprising CD20+CD19 binding domains with CD28 and 41BB co-stimulatary domains.

FIG. 3 provides schematic diagram of CAR construct comprising CD20+CD19 binding domains with ICOS, OX40 and 41BB co-stimulatary domains.

FIG. 4 provides schematic diagram of CAR construct comprising CD22+CD19 binding domains with OX40 and 41BB co-stimulatary domains.

FIG. 5 provides schematic diagram of CAR construct comprising CD22+CD19 binding domains with ICOS, OX40 and 41BB co-stimulatary domains.

FIG. 6 provides schematic diagram of CAR construct comprising CD22+CD19 binding domains with CD28 and 41BB co-stimulatary domains.

FIG. 7 provides schematic diagram of CAR construct comprising CD20+CD19 binding domains with ICOS and OX40 co-stimulatary domains.

FIG. 8 provides schematic diagram of CAR construct comprising CD22+CD19 binding domains with ICOS and OX40 co-stimulatary domains.

FIG. 9 provides schematic diagram of CAR construct comprising CD20+CD19 binding domains with ICOS and 41BB co-stimulatary domains.

FIG. 10 provides schematic diagram of CAR construct comprising CD22+CD19 binding domains with ICOS and 41BB co-stimulatary domains.

FIG. 11 provides digestion confirmation representative results (CD28/41BB). wherein A represents digestion image of Fsel+Kpn1 and B represents digestion image of SgrA1+Apa1 and C represents digestion image of SgrA1+Nhe1 for all 5 scFv combinations of CD28/41BB constructs.

FIG. 12 shows Sanger Sequencing Representative Results (CD28/41BB). Panel A: Representative Image of the synthetic CAR construct containing EF1 alpha promoter in the scFv region, either CD22 or CD20 which is joined to CD19 via (G4S)4 linker, in the trans-membrane region, a CD8 domain and in the co stimulatory region, CD28 and 41BB followed by a CD3 zeta co-signaling domain. Panel B: Screenshot of chromatogram for sanger sequencing showing the co-stimulatory domain region with clear and single peaks and high quality score. Panel C: ClustalW Alignment of sanger result with reference file spanning the costimulatory region.

FIG. 13 shows Sanger Sequencing Representative Results (OX40/41BB). Panel A: Representative Image of the synthetic CAR construct containing EF1 alpha promoter, in the scFv region, either CD22 or CD20 which is joined to CD19 via (G4S)4 linker, in the trans-membrane region, a CD8 domain and in the co stimulatory region, OX40 and 41BB followed by a CD3 zeta co signaling domain. Panel B: Screenshot of chromatogram for sanger sequencing showing the co-stimulatory domain region with clear and single peaks and high quality score. Panel C: ClustalW Alignment of sanger result with reference file spanning the costimulatory region

FIG. 14 shows Sanger Sequencing Representative Results (ICOS/41BB). Panel A: Representative Image of the synthetic CAR construct containing EF1 alpha promoter, in the scFv region, either CD22 or CD20 which is joined to CD19 via (G4S)4 linker, in the trans-membrane region, a CD8 domain and in the co stimulatory region. ICOS and 41BB followed by a CD3 zeta co signaling domain. Panel B: Screenshot of chromatogram for sanger sequencing showing the co-stimulatory domain region with clear and single peaks and high quality score. Panel C: ClustalW Alignment of sanger result with reference file spanning the costimulatory region.

FIG. 15 shows Sanger Sequencing Representative Results (ICOS/OX40). Panel A: Representative Image of the synthetic CAR construct containing EF1 alpha promoter, in the scFv region, either CD22 or CD20 which is joined to CD19 via (G4S)4 linker, in the trans-membrane region, a CD8 domain and in the co stimulatory region, ICOS and OX40 followed by a CD3 zeta co signaling domain. Panel B: Screenshot of chromatogram for sanger sequencing showing the co-stimulatory domain region with clear and single peaks and high quality score. Panel C: ClustalW Alignment of sanger result with reference file spanning the costimulatory region. The primers for sanger sequencing were chosen such that that each region is completely covered and the ends are overlapped in order to cancel out bad end-reads. 7 primers named 1F, 2AF, 2BF, 2CF, 3F, 4R and 5F were used.

FIG. 16 shows Sanger Sequencing Representative Results (ICOS/OX40/41BB). Panel A: Representative Image of the synthetic CAR construct containing EF1 alpha promoter, in the scFv region, either CD22 or CD20 which is joined to CD19 via (G4S)4 linker, in the trans-membrane region, a CD8 domain and in the co stimulatory region, ICOS, OX40 and 41BB followed by a CD3 zeta co signaling domain. Panel B: Screenshot of chromatogram for sanger sequencing showing the co-stimulatory domain region with clear and single peaks and high quality score. Panel C: ClustalW Alignment of sanger result with reference file spanning the costimulatory region. The primers for sanger sequencing were chosen such that that each region is completely covered and the ends are overlapped in order to cancel out bad end-reads. 7 primers named 1F, 2AF, 2BF, 2CF, 3F, 4R and 5F were used.

FIG. 17 shows flow cytometric analysis of expression of various CAR constructs in T cells. The histogram A shows the expression of various constructs with either a single co-stimulatory domain (4-1BB) or more than one (4-1BB and CD28). The histogram B shows the expression of various constructs with either a single co-stimulatory domain (4-1BB) or any two of ICOS 4-1BB and CD28. The antibodies were directed to recognise CD19 scFv, CD20 or CD22 scFv domains.

FIG. 18 shows results of bioluminance assay of Tandem, bispecific 3^{rd} gen CAR T with CD28 and 4-1BB domains illustrating percentage of antigen specific cell death. The Panel A shows bispecific CD20/CD19 CAR T-cells and Panel B shows CD22/CD19 CAR T cells; both containing CD28 and 4-1BB as co-stimulatory domains. The X-axis indicates E (effector): T (target) cell ratio.

FIG. 19 shows results of bioluminance assay of Tandem, bispecific 3^{rd} gen CAR T with ICOS and 4-1BB domains illustrating percentage of antigen specific cell death. The Panel A shows bispecific CD20/CD19 CAR T-cells and Panel B shows CD22/CD19 CAR T cells; both containing ICOS and 4-1BB as co-stimulatory domains. The X-axis indicates E (effector): T (target) cell ratio.

FIG. 20 shows CAR T cell proliferation rate of CD22/CD19 or CD20/CD19 CAR T cells expressing two co-stimulatory domains and CD19 CAR T cells with a single co-stimulatory domain.

FIG. 21 shows CAR T cell activation rate of CD22/CD19 or CD20/CD19 CAR T cells expressing two co-stimulatory domains and CD19 CAR T cells with a single co-stimulatory domain.

### SUMMARY OF THE INVENTION

### Technical Problem

The technical problem to be solved by the present invention is to provide effective CAR-T cell therapies which can be used for targeting B cell malignancies and decrease the chance of relapse or recurrence of cancer.

### The Solution to the Problem

The problem has been solved by developing bi-specific 3^{rd} and 4^{th} generation chimeric antigenic receptors (CARs) with a novel combination of co-stimulatory domains. In particular, the CAR of the present invention can effectuate high levels of cytokine secretion and considerably increase proliferation speed and survival rate of engrafted T-cells.

The present invention relates to a bi-specific chimeric antigenic receptor (CARs) with a novel combination of two or more co-stimulatory domains.

The present invention also relates to a viral vector comprising a nucleic acid encoding the said CAR.

The present invention further relates to the use of said CAR in treating B-cell related malignancies.

### BRIEF DESCRIPTION OF SEQUENCE LISTING

The sequences of the 3^{rd} and 4^{th} generation chimeric antigen receptors of the present invention are briefly described herein:

| Sequence ID | |
|---|---|
| Number | Brief Description of Sequences |
| SEQ ID NO: 1 | Nucleotide Sequence of CAR Construct comprising CD20 and CD19 binding |
| | domains with OX40 and 41BB co-stimulatory domains |
| SEQ ID NO: 2 | Nucleotide Sequence of CAR Construct comprising CD20 and CD19 binding |
| | domains with CD28 and 41BB co-stimulatory domains |
| SEQ ID NO: 3 | Nucleotide Sequence of CAR Construct comprising CD20 and CD19 binding |
| | domains with ICOS, OX40 and 41BB co-stimulatory domains |
| SEQ ID NO: 4 | Nucleotide Sequence of CAR Construct comprising CD19 and CD22 binding |
| | domains with OX40 and 41BB co-stimulatory domains |
| SEQ ID NO: 5 | Nucleotide Sequence of CAR Construct comprising CD232 and CD19 binding |
| | domains with ICOS, OX40 and 41BB co-stimulatory domains |
| SEQ ID NO: 6 | Nucleotide Sequence of CAR Construct comprising CD22 and CD19 binding |
| | domains with CD28 and 41BB co-stimulatory domains |
| SEQ ID NO: 7 | Nucleotide Sequence of CAR Construct comprising CD20 and CD19 binding |
| | domains with ICOS and OX40 co-stimulatory domains |
| SEQ ID NO: 8 | Nucleotide Sequence of CAR Construct comprising CD22 and CD19 binding |
| | domains with ICOS and OX40 co-stimulatory domains |
| SEQ ID NO: 9 | Nucleotide Sequence of CAR Construct comprising CD20 and CD19 binding |
| | domains with ICOS and 41BB co-stimulatory domains |
| SEQ ID NO: 10 | Nucleotide Sequence of CAR Construct comprising CD22 and CD19 binding |
| | domains with ICOS and 41BB co-stimulatory domains |
| SEQ ID NO: 11 | Amino acid sequence of CAR of Example 1 |
| SEQ ID NO: 12 | Amino acid sequence of CAR of Example 2 |
| SEQ ID NO: 13 | Amino acid sequence of CAR of Example 3 |
| SEQ ID NO: 14 | Amino acid sequence of CAR of Example 4 |
| SEQ ID NO: 15 | Amino acid sequence of CAR of Example 5 |
| SEQ ID NO: 16 | Amino acid sequence of CAR of Example 6 |
| SEQ ID NO: 17 | Amino acid sequence of CAR of Example 7 |
| SEQ ID NO: 18 | Amino acid sequence of CAR of Example 8 |
| SEQ ID NO: 19 | Amino acid sequence of CAR of Example 9 |
| SEQ ID NO: 20 | Amino acid sequence of CAR of Example 10 |
| SEQ ID NO: 21 | Amino acid sequence of CD19 binding domain of CAR |
| SEQ ID NO: 22 | Amino acid sequence of CD20 binding domain of CAR |
| SEQ ID NO: 23 | Amino acid sequence of CD22 binding domain of CAR |
| SEQ ID NO: 24 | Amino acid sequence of (G4S)₅-linker |
| SEQ ID NO: 25 | Amino acid sequence of CD8 hinge of CAR |
| SEQ ID NO: 26 | Amino acid sequence of CD8 transmembrane domain of CAR |
| SEQ ID NO: 27 | Amino acid sequence of ICOS transmembrane domain of CAR |
| SEQ ID NO: 28 | Amino acid sequence of ICOS co-stimulatory domain of CAR |
| SEQ ID NO: 29 | Amino acid sequence of OX40 co-stimulatory domain of CAR |
| SEQ ID NO: 30 | Amino acid sequence of 41BB co-stimulatory domain of CAR |
| SEQ ID NO: 31 | Amino acid sequence of CD28 co-stimulatory domain of CAR |
| SEQ ID NO: 32 | Amino acid sequence of CD3ζ domain of CAR |

### DETAILED DESCRIPTION OF THE INVENTION

Embodiments described herein can be understood more readily by reference to the following detailed description, examples, and drawings. Elements and methods described herein are merely illustrative of the principles of the present invention and are not limited to the specific embodiments presented in the detailed description, examples, and drawings. Numerous modifications and adaptations will be readily apparent to those of skill in the art without departing from the spirit and scope of the invention.

Before the methods and products of the present disclosure are described in greater detail, it is to be understood that the invention is not limited to particular embodiments and 10 may vary. It is also to be understood that the terminology used herein is for the purpose of describing particular embodiments only, and is not intended to be limiting, since the scope of the methods and devices will be limited only by the appended claims.

The present invention discloses bi-specific 3^{rd} and 4^{th} generation chimeric antigenic receptors (CARs) with a novel combination of co-stimulatory domains.

The invention contemplates a multidimensional approach in the development of highly effective chimeric antigenic receptors which can be used for targeting B cell malignancies such as Acute Lymphoblastic Leukemia and B cell lymphomas. The invention overcomes the problems of the prior art, specifically 2^{nd} generation chimeric antigenic receptors, and is particularly useful in exhibiting high efficacy against B-cell malignancies. Further, the risk of relapse or recurrence of cancer decreases with the usage of the chimeric antigenic receptors of the present invention.

Unless defined otherwise, all technical and scientific terms used herein have the same meaning as commonly understood by one of ordinary skill in the art to which the methods belong. Although any method and embodiments similar or equivalent to those described herein can also be used in the practice or testing of the methods and embodiments, representative illustrative methods and embodiments are now described.

Where a range of values is provided, it is understood that each intervening value between the upper and lower limit of that range and any other stated or intervening value in that stated range, is encompassed within by the methods. The upper and lower limits of these smaller ranges may independently be included in the smaller ranges and are also encompassed within by the methods, subject to any specifically excluded limit in the stated range. Where the stated range includes one or both of the limits, ranges excluding either or both of those included limits are also included in the methods.

It is appreciated that certain features of the methods, which are, for clarity, described in the context of separate embodiments, may also be provided in combination in a single embodiment. Conversely, various features of the methods and compositions, which are, for brevity, described in the context of a single embodiment, may also be provided separately or in any suitable sub-combination. It is noted that, as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements or use of a “negative” limitation.

As will be apparent to those of skill in the art upon reading this disclosure, each of the individual embodiments described and illustrated herein has discrete features which may be readily separated from or combined with the features of any of the other embodiments without departing from the scope or spirit of the present methods. Any recited method can be carried out in the order of events recited or in any other order that is logically possible.

The practice of the present invention will employ, unless indicated specifically to the contrary, conventional methods of virology, immunology, microbiology, molecular biology, and recombinant DNA techniques within the skill of the art. Such techniques are explained fully in the available literature.

Standard techniques may be used for recombinant DNA, oligonucleotide synthesis, and tissue culture and transformation. Enzymatic reactions and purification techniques may be performed according to the manufacturer's specifications or as commonly accomplished in the art or as described herein. These and related techniques and procedures may be generally performed according to conventional methods well known in the art.

As used herein, the terms, “binding domain” or “extracellular domain,” are used interchangeably and provide the CAR with the ability to bind to the target antigen of interest. A binding domain can be any protein, polypeptide, oligopeptide, or peptide that possesses the ability to specifically recognize and bind to a biological molecule e.g., a cell surface receptor or tumor protein, or a component thereof. A binding domain includes any naturally occurring, synthetic, semi-synthetic, or recombinantly produced binding partner for a biological molecule of interest.

The 3^{rd} and and 4^{th} generation bispecific CARs of the present invention are designed to contain at least two binding (extracellular) domains and at least two signaling/co-stimulatory (intracellular) domains.

In one embodiment, the 3^{rd} and 4^{th} generation chimeric antigen receptors of the present invention comprise four functional domains: (1) at least two binding domains that bind the antigen and thereby target the CAR-expressing immune effector cell to a target cell expressing CD19, CD20 or CD22; (2) a hinge or spacer region that extends the binding domain away from the effector cell plasma membrane: (3) a transmembrane domain that anchors the CAR to the effector cell and links the binding domain to the intracellular signaling domain; and (4) an intracellular domain comprising a signaling domain or domains, and optionally one or more co-stimulatory signaling domains.

In certain embodiments, suitable linkers are used for designing the CAR. The linker residues can be added between various domains for appropriate spacing and conformation of the molecule. Suitable linkers can be readily selected and can be of different suitable lengths, such as from 1 amino acid (e.g., Gly) to 20 amino acids, from 2 amino acids to 15 amino acids, from 3 amino acids to 12 amino acids, including 4 amino acids to 10 amino acids, 5 amino acids to 9 amino acids, 6 amino acids to 8 amino acids, or 7 amino acids to 8 amino acids, and maybe 1, 2, 3, 4, 5, 6, or 7 amino acids. Exemplary flexible linkers include glycine polymers (G), glycine-serine polymers, where n is an integer of at least one. The ordinarily skilled artisan will recognize that the design of a CAR can include linkers that are all or partially flexible, such that the linker can include a flexible linker as well as one or more portions that confer less flexible structure to provide for the desired CAR structure.

In one embodiment, the binding (extracellular) domains of the chimeric antigen receptors (CARs) are selected from a group comprising CD19, CD20 and CD22.

The binding domain of the CAR is generally followed by a “spacer,” or “hinge,” which refers to the region that moves the antigen-binding domain away from the effector cell surface to enable proper cell/cell contact, antigen binding and activation. The hinge region in a CAR is generally between the TM and the binding domain.

In certain embodiments, a hinge region is an immunoglobulin hinge region and may be a wild-type immunoglobulin hinge region or an altered wild-type immunoglobulin hinge region.

In another embodiment, the hinge or spacer region of the chimeric antigen receptors (CARs) is from CD8 hinge.

The “transmembrane,” region or domain is the portion of the CAR that anchors the extracellular binding portion to the plasma membrane of the immune effector cell and facilitates binding of the binding domain to the target antigen.

In another embodiment, the transmembrane domain of the chimeric antigen receptors (CARs) is selected from a group comprising CD8 transmembrane domain or ICOS transmembrane domain.

The “intracellular signaling domain,” refers to the part of the chimeric antigen receptor protein that participates in transducing the message of effective CAR binding to a target antigen into the interior of the immune effector cell to elicit effector cell function, e.g., activation, cytokine production, proliferation and cytotoxic activity, including the release of cytotoxic factors to the CAR-bound target cell, or other cellular responses elicited with antigen binding to the extracellular CAR domain.

Further, “co-stimulatory signaling domain,” or “co-stimulatory domain” are also included in the CAR design of the present invention. As used herein, “co-stimulatory domain” refers to the portion of the CAR comprising the intracellular domain of a co-stimulatory molecule. Co-stimulatory molecules are cell surface molecules other than antigen receptors or F_{c} receptors that provide a second signal required for efficient activation and function of T lymphocytes upon binding to antigen.

In one embodiment, the intracellular domains of the chimeric antigen receptors (CARs) are selected from a group comprising CD28 co-stimulatory domain, ICOS co-stimulatory domain, OX40 co-stimulatory domain, 41BB co-stimulatory domain, followed by CD3ζ domain.

In another embodiment, the invention provides 3^{rd} and and 4^{th} generation chimeric antigen receptors comprising the following functional domains: (1) at least two binding domains that bind the antigen and thereby target the CAR-expressing immune effector cell to a target cell expressing CD19, CD20 or CD22; (2) a GS linker region that links the two binding domains in a specific conformation; (3) a CD8 hinge or spacer domain that extends the binding domain away from the effector cell plasma membrane; (4) a CD8 or ICOS transmembrane domain that anchors the CAR to the effector cell and links the binding domain to the intracellular signaling domain; and (5) an intracellular domain comprising a CD3ζ (CD3-zeta) signaling domain, and (6) optionally one or more co-stimulatory signaling domains selected from a group comprising CD28 co-stimulatory domain, ICOS co-stimulatory domain, OX40 co-stimulatory domain, 41BB co-stimulatory domain.

In another embodiment, the invention provides a chimeric antigen receptor comprising CD19 and CD20 binding domains (scFv), a GS linker, a hinge and transmembrane domain from CD8 domain, and co-stimulatory domains OX40 and 41BB, followed by intracellular signaling CD3ζ domain.

In another embodiment, the invention provides a chimeric antigen receptor comprising CD19 and CD20 binding domains (scFv), a GS linker, a hinge from CD8 domain, and co-stimulatory domains CD28 and 41BB, followed by intracellular signaling CD3ζ domain.

In another embodiment, the invention provides a chimeric antigen receptor comprising CD19 and CD20 binding domains (scFv), a GS linker, a hinge from CD8 domain, a transmembrane domain from ICOS and co-stimulatory domains ICOS, OX40 and 41BB, followed by intracellular signaling CD3ζ domain.

In another embodiment, the invention provides a chimeric antigen receptor comprising CD19 and CD22 binding domains (scFv), a GS linker, a hinge and a transmembrane domain from CD8 and co-stimulatory domains OX40 and 41BB, followed by intracellular signaling CD3ζ domain.

In another embodiment, the invention provides a chimeric antigen receptor comprising CD19 and CD22 binding domains (scFv), a GS linker, a hinge from CD8, a transmembrane domain from ICOS and co-stimulatory domains ICOS, OX40 and 41BB, followed by intracellular signaling CD3ζ domain.

In another embodiment, the invention provides a chimeric antigen receptor comprising CD19 and CD22 binding domains (scFv), a GS linker, a hinge from CD8, and co-stimulatory domains CD28 and 41BB, followed by intracellular signaling CD3ζ domain.

In another embodiment, the invention provides a chimeric antigen receptor comprising CD20 and CD19 binding domains (scFv), a GS linker, a hinge from CD8, transmembrane domain from ICOS and co-stimulatory domains ICOS and OX40, followed by intracellular signaling CD3ζ domain.

In another embodiment, the invention provides a chimeric antigen receptor comprising CD22 and CD19 binding domains (scFv), a GS linker, a hinge from CD8, transmembrane domain from ICOS and co-stimulatory domains ICOS and OX40, followed by intracellular signaling CD3ζ domain.

In another embodiment, the invention provides a chimeric antigen receptor comprising CD20 and CD19 binding domains (scFv), a hinge from CD8, transmembrane domain from ICOS and co-stimulatory domains ICOS and 41BB, followed by intracellular signaling CD3ζ domain.

In another embodiment, the invention provides a chimeric antigen receptor comprising CD22 and CD19 binding domains (scFv), a hinge from CD8, transmembrane domain from ICOS and co-stimulatory domains ICOS and 41BB, followed by intracellular signaling CD3ζ domain.

In another embodiment, the invention provides a linker region selected from the group consisting of (G2S)₃-linker; (G3S)₃-linker, (G4S)₃-linker, (G2S)₄-linker, (G3S)₄-linker, (G4S)₄-linker, (G2S)₅-linker, (G3S)₅-linker, or (G4S)₅-linker.

In another embodiment, the invention relates to a nucleic acid or nucleic acid construct encoding a chimeric antigen receptor, the chimeric protein comprising several polypeptide portions: (1) a binding domain that binds CD19, CD20 or CD22, e.g., an anti-CD19 antibody, anti-CD20 antibody, anti-CD22 antibody, or an antigen-binding fragment thereof (such as a scFv derived from a murine, human or humanized antibody that binds CD19, CD20 or CD22); (2) a GS linker region; (3) a CD8 hinge or spacer region; (4) a CD8 or ICOS transmembrane region and (5) a human CD3ζ intracellular signaling domain, and (6) optionally one or more intracellular co-stimulatory signaling domains selected from the group consisting of CD28, 41BB, OX40 and ICOS.

In another embodiment, the invention relates to an immune effector cell or progenitor of an immune effector cell, comprising one or more of the CAR proteins described herein and a population of immune effector cells that have been modified to express one or more of the CAR proteins described herein.

In one embodiment, the immune effector cells are T cells, NK cells, NKT cells, or those mature immune effector cells including neutrophils, macrophages arising from CAR-modified hematopoietic stem cells (HSC) within the population of cells present in cord blood, bone marrow or mobilized peripheral blood.

In another embodiment, the invention relates to a method of making and expanding CD19, CD20 or CD22-specific CAR immune effector cells which comprises introducing into immune effector cells, an expression vector containing a nucleic acid construct encoding a CD19, CD20 or CD22-specific CAR as described herein, stimulating the cells with antibodies against CD19, CD20 or CD22 in the presence of IL-2, or tumor targets expressing CD19, CD20 or CD22 antigen in the presence of IL-2.

In another embodiment, the invention provides a vector comprising the nucleic acid or nucleic acid construct encoding a chimeric antigen receptor of the present invention.

In another embodiment, the present invention relates to a vector comprising a nucleic acid encoding a CD19, CD20 or CD22-specific CAR as described herein.

In another embodiment, the vector is an expression vector.

In another embodiment, the expression vector is selected from the group consisting of lentiviral or retroviral vectors.

In another embodiment, the vector may comprise of CMV promoter/enhancer, a Psi (Ψ) packaging signal, a central polypurine tract/central termination sequence (cPPT/CTS), EF1 alpha promoter for optimal expression in T cells, a linker/hinge linked to the binding domain, woodchuck hepatitis virus posttranscriptional regulatory element (WPRE), f1 ori and promoter for ampicillin resistance.

In another embodiment, a transgene consisting the CAR cassette is under the control of the EF1 alpha promoter for optimal expression in T cells.

The present disclosure also provides methods to stably introduce and redirect immune effector cells by viral transduction.

In another embodiment, the invention provides immune effector cell comprising the vector as described herein.

In another embodiment, the invention provides a composition comprising the immune effector cell and optionally, a pharmaceutically acceptable excipient.

In another embodiment, the pharmaceutically acceptable excipients may comprise of buffer for optimal, sodium chloride, plasma lyte, dextran, dextrose, glycerol, maltose, sucrose or a combination of various buffers.

In another embodiment, the present invention relates to a method of treating a B cell related condition in a subject in need thereof, comprising administering to the subject a therapeutically effect amount of the composition.

In another embodiment, the B cell related condition is Acute Lymphoblastic Leukemia, B cell lymphomas, multiple myeloma, non-Hodgkin's lymphoma, B cell proliferations of lymphomatoid uncertain malignant potential, granulomatosis, post-transplant lymphoproliferative disorder, mantle cell lymphoma, follicular lymphoma, or Burkitt lymphoma.

In another embodiment, the B cell related condition is a B cell malignancy.

In another embodiment, the B cell malignancy is Acute Lymphoblastic Leukemia and

B cell lymphomas.

In another embodiment, the CAR or the polynucleotide encoding a CAR is use in treating B-cell malignancy.

In another embodiment, the invention provides a method of generating immune effector cells comprising introducing into an immune effector cell, the vectors as described herein, stimulating the cells and inducing the cells to proliferate by contacting the cells in the presence of IL2 or a combination of IL-7 and IL-15 with antibodies that bind to the extracellular domains and antibodies that bind to the extracellular domains; thereby generating the specific immune effector cell.

Other embodiments provide a variety of methods for genetically modifying a cell with a vector that comprises a nucleic acid encoding an anti-CD19 antibody, anti-CD20 antibody, anti-CD22 antibody CAR as described herein.

Such vectors and methods of genetically modifying cells (e.g., immune effector cells) include a variety of expression vectors and methods of introducing such vectors into cells, such as transfection, electroporation, transduction, gene gun, and the like.

Thus, in another embodiment, the invention relates to a method of stably introducing and re-directing immune effector cells by electroporation using naked DNA or introduction of DNA using chemical transfection reagents and related compounds known in the art.

In another embodiment, the invention relates to methods of treating a human with a B cell malignancy comprising administering to a human diagnosed with said malignancy, a population of modified human immune effector cells or a population of progenitors of immune effector cells that upon administration can differentiate into immune effector cells, expressing the CARs described herein.

In another embodiment, the invention relates to a method of treating a subject with a B cell malignancy comprising removing immune effector cells such as T, NK, NKT, or hematopoietic stem cells (HSCs), introducing into said immune effector cells a vector comprising a nucleic acid encoding the CAR proteins described herein, and administering the population of said immune effector cells to the same subject.

In certain embodiments, the removed immune effector cells are not expanded before modifying with CAR expression vector.

In other embodiments, CAR-T cells are pre-stimulated with at least one cytokines, followed by exposure to the viral vector prior to re-administration into a subject.

In particular embodiments, CAR-T cells of the present invention are used for the treatment of B cell malignancies such as Acute Lymphoblastic Leukemia and B cell lymphomas.

In an embodiment, CAR-T cells of the present invention are used for the treatment of B cell related condition such as Acute Lymphoblastic Leukemia, B cell lymphomas, multiple myeloma, non-Hodgkin's lymphoma, B cell proliferations of uncertain malignant potential, lymphomatoid granulomatosis, post-transplant lymphoproliferative disorder, mantle cell lymphoma, follicular lymphoma, or Burkitt lymphoma.

Non-limiting examples that depict the various embodiments of the 3rd and and 4th generation chimeric antigen receptors of the present invention are provided herein:

### Example 1: Chimeric Antigen Receptor Comprising CD20 and CD19 Binding Domains With OX40 and 41BB co-Stimulatory Domains

3^{rd} generation chimeric antigen receptor has been designed to contain CD19 and CD20 binding domains (scFv), a hinge and transmembrane domain from CD8, and co-stimulatory domains OX40 and 41BB, followed by intracellular signaling CD3ζ domain. Optionally, a glycine-serine (G4S)₅-linker may be used for appropriate spacing and conformation of the CAR.

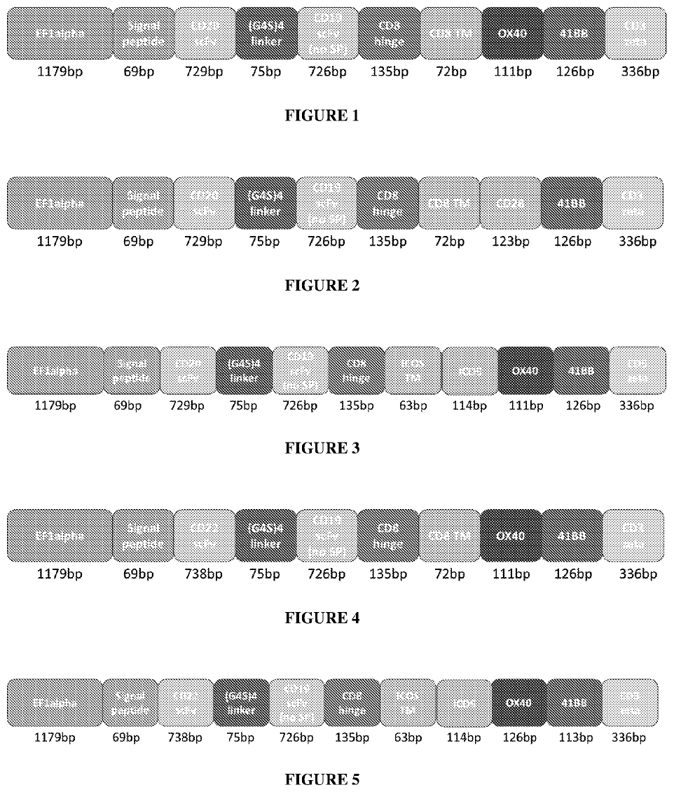

The CAR construct is provided in FIG. 1 and the nucleotide sequence of the CAR construct is provided in SEQ ID NO: 1. SEQ ID NO: 11 provides the amino acid sequence of CAR of Example 1.

### Example 2: Chimeric Antigen Receptor Comprising CD20 and CD19 Binding Domains With CD28 and 41BB co-Stimulatory Domains

3^{rd} generation chimeric antigen receptor has been designed to contain CD20 and CD19 binding domains (scFv), a hinge and transmembrane domain from CD8, and co-stimulatory domains CD28 and 41BB, followed by intracellular signaling CD3ζ domain. Optionally, a glycine-serine (G4S)₅-linker may be used for appropriate spacing and conformation of the CAR.

The CAR construct is provided in FIG. 2 and the nucleotide sequence of the CAR construct is provided in SEQ ID NO: 2. SEQ ID NO: 12 provides the amino acid sequence of CAR of Example 2.

### Example 3: Chimeric Antigen Receptor Comprising CD20 and CD19 Binding Domains With ICOS, OX40 and 41BB co-Stimulatory Domains

4^{th} generation chimeric antigen receptor has been designed to contain CD20 and CD19 binding domains (scFv), a hinge from CD8, a transmembrane domain from ICOS and co-stimulatory domains ICOS, OX40 and 41BB, followed by intracellular signaling CD3ζ domain. Optionally, a glycine-serine (G4S)₅-linker may be used for appropriate spacing and conformation of the CAR.

The CAR construct is provided in FIG. 3 and the nucleotide sequence of the CAR construct is provided in SEQ ID NO: 3. SEQ ID NO: 13 provides the amino acid sequence of CAR of Example 3.

### Example 4: Chimeric Antigen Receptor Comprising CD22 and CD19 Binding Domains With OX40 and 41BB co-Stimulatory Domains

3^{rd} generation chimeric antigen receptor has been designed to contain CD22 and CD19 binding domains (scFv), a hinge and a transmembrane domain from CD8 and co-stimulatory domains OX40 and 41BB, followed by intracellular signaling CD3ζ domain. Optionally, a glycine-serine (G4S)₅-linker may be used for appropriate spacing and conformation of the CAR.

The CAR construct is provided in FIG. 4 and the nucleotide sequence of the CAR construct is provided in SEQ ID NO: 4. SEQ ID NO: 14 provides the amino acid sequence of CAR of Example 4.

### Example 5: Chimeric Antigen Receptor Comprising CD22 and CD19 Binding Domains With ICOS, OX40 and 41BB co-Stimulatory Domains

4^{th} generation chimeric antigen receptor has been designed to contain CD22 and CD19 binding domains (scFv), a hinge from CD8, a transmembrane domain from ICOS and co-stimulatory domains ICOS, OX40 and 41BB, followed by intracellular signaling CD3ζ domain. Optionally, a glycine-serine (G4S)₅-linker may be used for appropriate spacing and conformation of the CAR.

The CAR construct is provided in FIG. 5 and the nucleotide sequence of the CAR construct is provided in SEQ ID NO: 5. SEQ ID NO: 15 provides the amino acid sequence of CAR of Example 5.

### Example 6: Chimeric Antigen Receptor Comprising CD22 and CD19 Binding Domains With CD28 and 41BB co-Stimulatory Domains

3^{rd} generation chimeric antigen receptor has been designed to contain CD22 and CD19 binding domains (scFv), a hinge and transmembrane domain from CD8, and co-stimulatory domains CD28 and 41BB, followed by intracellular signaling CD3ζ domain. Optionally, a glycine-serine (G4S)₅-linker may be used for appropriate spacing and conformation of the CAR.

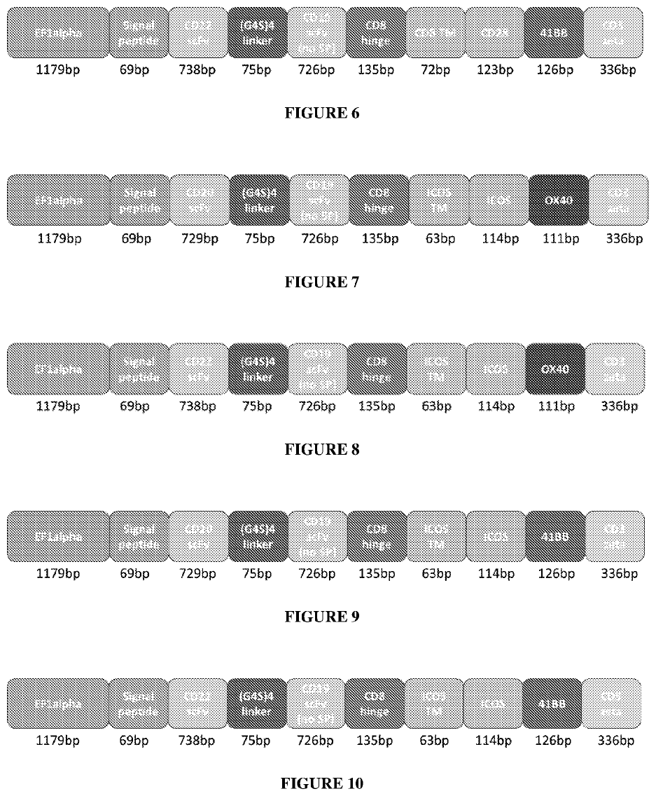

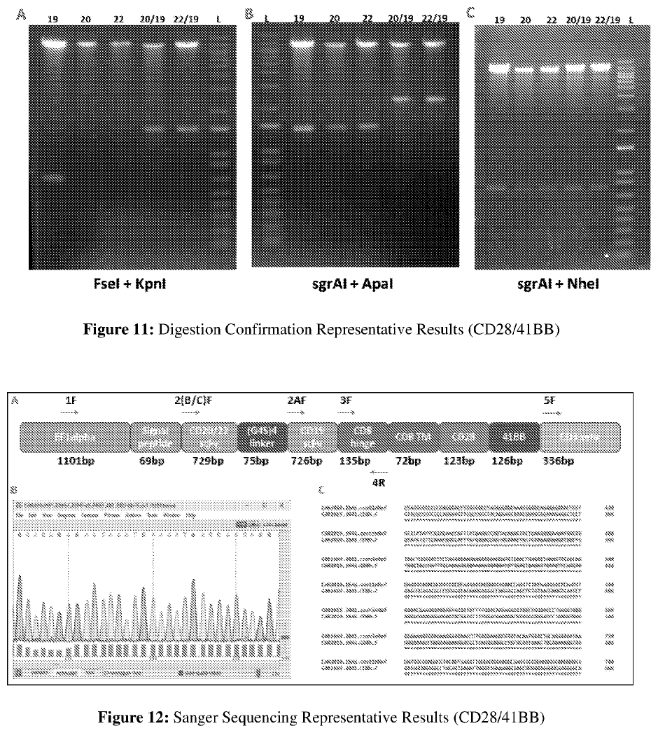

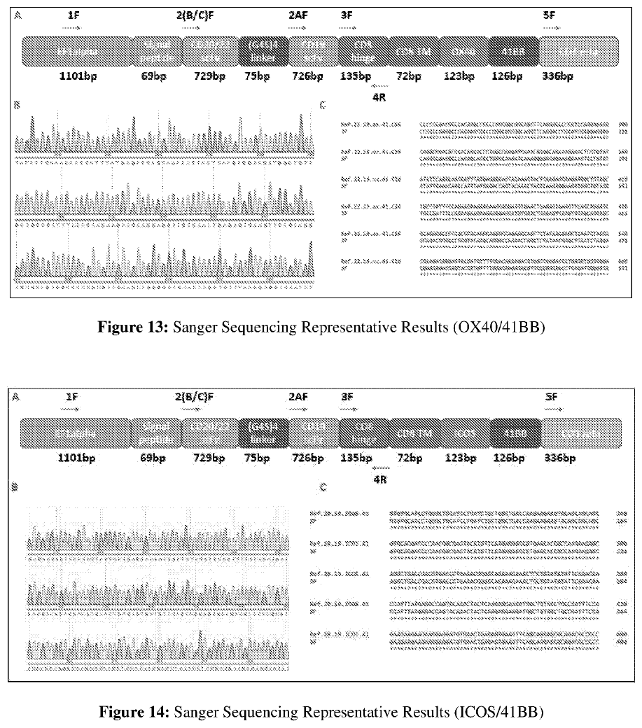

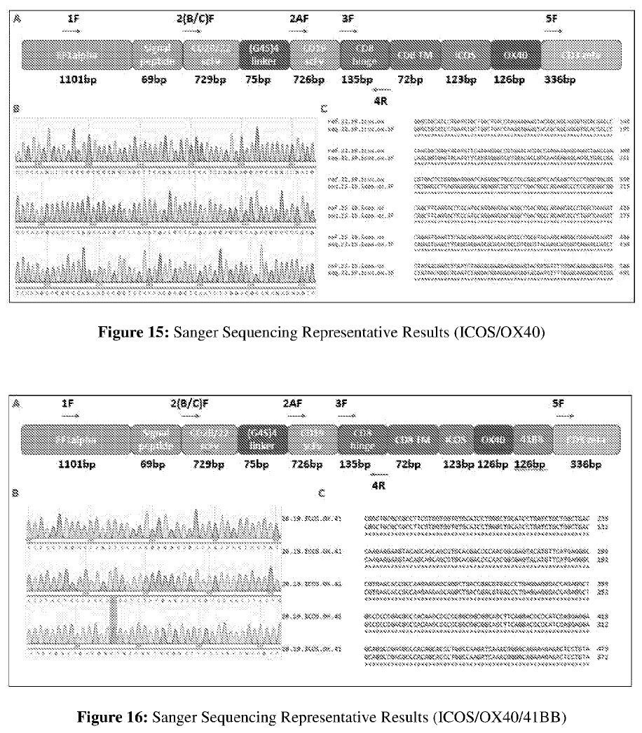

The CAR construct is provided in FIG. 6 and the nucleotide sequence of the CAR construct is provided in SEQ ID NO: 6. SEQ ID NO: 16 provides the amino acid sequence of CAR of Example 6.

### Example 7: Chimeric Antigen Receptor Comprising CD20 and CD19 Binding Domains With ICOS and OX40 co-Stimulatory Domains

3^{rd} generation chimeric antigen receptor has been designed to contain CD20 and CD19 binding domains (scFv), a hinge from CD8, transmembrane domain from ICOS, and co-stimulatory domains ICOS and OX40, followed by intracellular signaling CD3ζ domain. Optionally, a glycine-serine (G4S)₅-linker may be used for appropriate spacing and conformation of the CAR.

The CAR construct is provided in FIG. 7 and the nucleotide sequence of the CAR construct is provided in SEQ ID NO: 7. SEQ ID NO: 17 provides the amino acid sequence of CAR of Example 7.

### Example 8: Chimeric Antigen Receptor Comprising CD22 and CD19 Binding Domains With ICOS and OX40 co-Stimulatory Domains

3^{rd} generation chimeric antigen receptor has been designed to contain CD22 and CD19 binding domains (scFv), a hinge from CD8, transmembrane domain from ICOS, and co-stimulatory domains ICOS and OX40, followed by intracellular signaling CD3ζ domain. Optionally, a glycine-serine (G4S)₅-linker may be used for appropriate spacing and conformation of the CAR.

The CAR construct is provided in FIG. 8 and the nucleotide sequence of the CAR construct is provided in SEQ ID NO: 8. SEQ ID NO: 18 provides the amino acid sequence of CAR of Example 8.

### Example 9: Chimeric Antigen Receptor Comprising CD20 and CD19 Binding Domains With ICOS and 41BB co-Stimulatory Domains

3^{rd} generation chimeric antigen receptor has been designed to contain CD20 and CD19 binding domains (scFv), a hinge from CD8, transmembrane domain from ICOS, and co-stimulatory domains ICOS and 41BB, followed by intracellular signaling CD3ζ domain. Optionally, a glycine-serine (G4S)₅-linker may be used for appropriate spacing and conformation of the CAR.

The CAR construct is provided in FIG. 9 and the nucleotide sequence of the CAR construct is provided in SEQ ID NO: 9. SEQ ID NO: 19 provides the amino acid sequence of CAR of Example 9.

### Example 10: Chimeric Antigen Receptor Comprising CD22 and CD19 Binding Domains With ICOS and 41BB co-Stimulatory Domains

3^{rd} generation chimeric antigen receptor has been designed to contain CD20 and CD19 binding domains (scFv), a hinge from CD8, transmembrane domain from ICOS, and co-stimulatory domains ICOS and 41BB, followed by intracellular signaling CD3ζ domain. Optionally, a glycine-serine (G4S)₅-linker may be used for appropriate spacing and conformation of the CAR.

The CAR construct is provided in FIG. 10 and the nucleotide sequence of the CAR construct is provided in SEQ ID NO: 10. SEQ ID NO: 20 provides the amino acid sequence of CAR of Example 10.

### Example 11: Construction and Expression of CARs

### 11.1 Generation of Stable Reporter Cancer Cell Lines

Raji cells that could stably express Luciferase were generated. The stable cells were generated by lentiviral mediated gene delivery of the transgene containing luciferase and mCherry. Single clones were made by first performing the FACS using mCherry and then by serial dilution method. Similarly, K562 cells which does not express CD19 and CD22 were also generated for the control experiments. The cell lines were used for the experiment and were purchased from ATCC and cultured as per the recommended protocol in RPMI-1640 supplemented with 10% FCS and Glutamax. The cell lines were maintained in the culture conditions with proper CO₂ levels and temperature.

### 11.2 Generation of CAR Constructs

CAR based plasmids were generated using DNA synthesis method and PCR for amplification. As discussed earlier, the CAR design consists of an scFV, a hinge region, a transmembrane domain based on CD8, at least two co-stimulatory molecules in various combinations selected from CD28, ICOS, 4-1BB and OX40 and a co-signaling molecule CD3ζ (CD3-zeta). The CAR constructs after synthesis were sub-cloned in the lentiviral vectors. General molecular cloning protocol was followed and reagents were obtained from Life Technologies.

The cloning of tandem CAR constructs was done using 3^{rd} generation lentiviral expressing pLenti-CD19 vector with CD28 and 41BB as co-stimulatory domains (pLenti-CD19-CD28-41BB). 1.5 Kb fragments of CD22-CD19 and CD20-CD19 were synthesized in which both the scFvs were separated by a (G4S)4 sequence. The synthesis was done such that Fsel and Kpn1 restriction enzyme sites were flanked on either side of the desired fragment. These synthesized fragments were then digested with Fse1 and Kpn1 and cloned in Fse1 and Kpn1 digested pLenti-CD28-41BB linearized backbone in order to get pLenti-CD22-CD19-CD28-41BB construct and pLenti-CD20-CD19-CD28-41BB constructs.

Further, two of the costimulatory domains ICOS and OX40 were synthesized as oligos with appropriate overhangs. Desired combinations of co-stimulatory domains were PCR amplified and ligated using annealing PCR. The outer primer combinations used for annealing PCR was designed such that flanking sequences would contain restriction enzyme site of sgRA1 in forward primer and Nhel in reverse primer. This strategy was followed to ultimately get following co-stimulatory domain combinations: CD28/OX40, CD28/ICOS, CD28/41BB, OX40/41BB, ICOS/41BB, ICOS/OX40, ICOS/OX40/41BB.

sgRA1 and Nhe1 restriction enzymes were chosen because they were common for all co-stimulatory domains and also it removes the existing co-stimulatory domain in pLenti-CD19-CD28-41BB vector. Therefore, once all the combinations of co-stimulatory domains were annealed, they were digested with sgRA1 and Nhel and cloned in sgRA1 and Nhel digested pLenti-CD19, pLenti-CD22-CD19 and pLenti linearized backbone.

TABLE 1
Data showing fallout and backbone sizes for each
double digestion combination as shown in FIG. 11.

| | | Size of Insert and | Size of Insert and | Size of Insert and | |
|---|---|---|---|---|---|
| | | backbone after | backbone after | backbone after | |
| | | digestion | digestion | digestion | |
| Sr. | | sgrA1 + Nhe1 | sgrA1 + Apa1 | Kpn1 + Fse1 | Total size |
| No. | Construct | digestion | digestion | digestion | of plasmid |
| 1 | CAR19_28/41 | 761 + 8266 | 1386 + 7641 | 651 + 8376 | 9027 |
| 2 | CAR20_28/41 | 761 + 8269 | 1389 + 7641 | Linearized | 9030 |
| 3 | CAR22_28/41 | 761 + 8284 | 1404 + 7641 | Linearized | 9045 |
| 4 | CAR20/19_28/41 | 761 + 9070 | 2190 + 7641 | 1445 + 8376 | 9831 |
| 5 | CAR22/19_28/41 | 761 + 9085 | 2205 + 7641 | 1470 + 8376 | 9846 |

### 11.3 Generation of Lenti-Particles

HEK293T cells were obtained from ATCC and seeded in DMEM complete medium supplemented with 10% FBS in 10 cm tissue culture plates, and cells were incubated at 37° C., 5% CO₂ for ˜24 hours. A mixture of the third generation lenti-plasmids and CAR plasmid were added into the cells using PEI as the transfection reagent. Cells were incubated for 48 hours before harvesting the medium containing the viral particles at different intervals, for upto 72 hours. The cell culture supernatant was centrifuged at 1000 rpm for 5 minutes to remove any packaging cell and debris. Supernatant was filtered using 0.45 μm PES filter. The viral particles were collected immediately. The viral particles were also stored at −80° C. for long term to avoid loss of titer.

### Example 12: Expression of Various CAR Constructs in T Cells

### 12.1 Peripheral Blood Mononuclear Cell (PBMC) Isolation

Fresh blood was drawn from the volunteers in presence of heparin as anti-coagulant. Blood collected was diluted with Dulbecco's Phosphate Buffered Saline (PBS). The diluted samples was subjected to density gradient separation using Ficoll 400 (Sigma-Aldrich), and the samples were centrifuged. After centrifugation, the PBMC layer was formed in the centre which was collected for further use.

### 12.2 Isolation of CD4+and CD8+T Lymphocytes

T lymphocytes were isolated from PBMCs using CD4 and CD8 isolation kits (Miltenyi Biotech). The isolation of CD4⁺ and CD8⁺T lymphocytes was done using the magnetic bead isolation method by performing the simultaneous isolation with equal ratios of beads. Purified cells were subjected to flow cytometry analysis. The cells were cultured in RPMI-1640 with 10% FBS. Primary T cells were cultured in human T cell medium consisting of X-VIVO 15 (Lonza), 5% Human AB serum, and 10 mM neutralized N-acetyl L-Cysteine (Sigma-Aldrich). Further, IL-2 was used for cell proliferation and 30 units/mL of IL-2 was used for all experiments.

### 12.3 Lentiviral Transduction to Express CARs

The isolated cells were stimulated with Human T-Activator CD3/CD28 Dynabeads (Life Technologies) at a 1:3 cell: bead ratio. At 24 hours, the primary T cells were exposed to viral supernatant (harvested as above). The cells were expanded until day 9 when they were rested and used for the assays.

### 12.4 Flow-Cytometric Analysis

CAR expression was performed using Flow cytometry. The cells were labelled with scFv CD19 and CD20 CAR detection reagents from Miltenyi biotech and CD20 CAR scFv detection reagent from Acrobiosystems. Analysis was performed using FlowJo software.

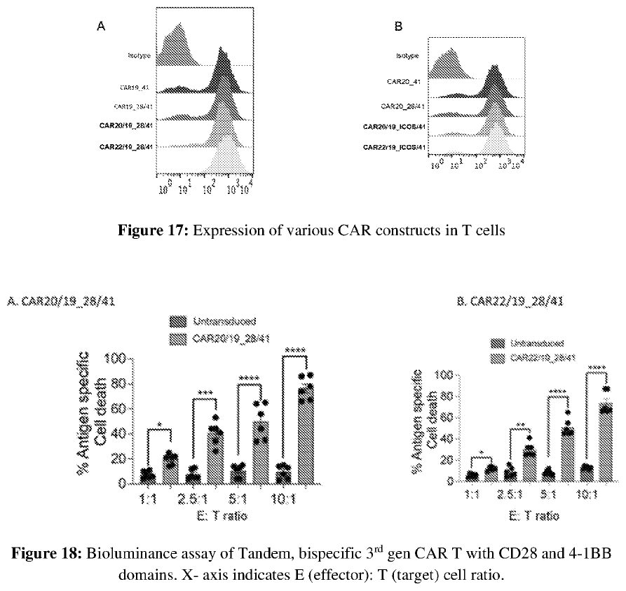

Flow cytometry analysis was done in T cells transduced with the various CAR T cell constructs to evaluate the expression. T cells were transduced with lentiviral particles of the respective CAR construct for 72 hours before the analysis was done. The histogram in FIG. 17 shows the expression of various constructs with either a single co-stimulatory domain (4-1BB) or more than one (4-1BB and CD28). Similarly, various other co-stimulatory domains were used such as ICOS in various combinations. The antibodies were directed to recognise CD19 scFv (A), CD20 (B), or CD22 (C) scFv domains. The data shown is the representation from n=3 independent experiments.

The data shown in panels A and B of FIG. 17 conclusively showed that the dual antigen expressing third generation CAR constructs were adequately expressed in T cells with respect to conventional second generation CAR constructs. The data further suggested that expression of two tumor antigen targeting ScFv domains (CD20/CD19 or CD22/CD19) does not interfere with their respective expression.

### 12.5 Luciferase-Based Cytolytic T-Cell Assay

Luciferase-based Cytolytic T-cell (CTL) assay is a standard and well-established method for checking cell death. Depending on the Tumor antigen type, this method can be optimized to be used against any cancer cell type or primary cells. In the present invention, Raji cells were used for stably expressing luciferase as the target cells. The CAR T cells expressing various scFv molecules and co-stimulatory domains were used for the co-culture experiments and the measurements were performed using bioluminance. Various effector to target cell ratios were used for the assay. The results for the assay were reported as percent killing based on luciferase activity in wells with tumor, but no T cells. (% killing=100−((RLU from well with effector and target cell co-culture)/(RLU from well with target cells)×100)). (Moon., et al 2014).

### Example 13: Bioluminance Assay of Tandem, Bispecific 3^{rd} Generation CAR T With CD28 and 4-1BB Domains

Luciferase expressing Raji were co-cultured with various CAR T cells for the indicated time points before the bioluminance assays was performed. The 3^{rd} generation tandem bispecific CD20/CD19 (A) or CD22/CD19 (B) CAR T cells containing CD28 and 4-1BB as co-stimulatory domains showed anti-tumor activity against the target antigen expressing Raji cells. Thus, these results confirm that the proposed CAR designs retain the anti-tumor activity. The CAR T to Raji cell ratio (E:T) used in this study was 1:1, 2.5:1, 1:5 and 10:1 and the cells were incubated for 72 hours before the assay was performed. The data shown in FIG. 18 is the representation from n=6 from three independent experiments.

The table 2 provides the comparative data showing percentage of antigen specific cell death in transduced and untransduced cells.

TABLE 2
Data showing percentage of antigen specific cell
death in transduced and untransduced cells for
two different constructs as shown in FIG. 18.

| | CD20/CD19_CD28 and | | CD22/CD19_CD28 and | |
|---|---|---|---|---|
| E:T | 4-1BB | | 4-1BB | |
| ratios | Untransduced | Transduced | Untransduced | Transduced |
| 1:1 | 5% | 20% | 5% | 15% |
| 2.5:1 | 5% | 40% | 10% | 30% |
| 5:1 | 10% | 50% | 10% | 50% |
| 10:1 | 10% | 80% | 15% | 70% |

The data shown in FIG. 18 and table 2 suggested that dual bi-specific CAR constructs possess potent anti-tumor activity against the target cancer cell lines. Notably, the co-expression of either CD20/CD19 or CD22/CD19 enhances the anti-tumor activity in comparison to the conventional second generation CAR T cells. Thus, third generation bi-specific CAR T cells of the present invention having CD28 and 4-1BB co-stimulatory domains are superior and have more potent anti-tumor killing potential than the conventional second generation mono-specific CAR T cells.

### Example 14: Bioluminance Assay of Tandem, Bispecific 3^{rd} Generation CAR T With ICOS and 4-1BB Domains

Luciferase expressing Raji were co-cultured with various CAR T cells for the indicated time points before the bioluminance assays was performed. The 3^{rd} generation tandem bispecific CD20/CD19 (A) or CD22/CD19 (B) CAR T cells containing ICOS and 4-1BB as co-stimulatory domains show anti-tumor activity against the target antigen expressing Raji cells. Thus, these results confirm that the proposed CAR designs retain the anti-tumor activity. The CAR T to Raji cell ratio (E:T) used in this study was 1:1, 2.5:1, 1:5 and 10:1 and the cells were incubated for 72 hours before the assay was performed. The data shown in FIG. 19 is the representation from n=6 from three independent experiments.

Table 3 provides the comparative data showing percentage of antigen specific cell death in transduced and untransduced cells.

TABLE 3
Data showing percentage of antigen specific cell
death in transduced and untransduced cells for
two different constructs as shown in FIG. 19.

| | CD20/CD19_ICOS and | | CD22/CD19_ICOS and | |
|---|---|---|---|---|
| E:T | 4-1BB | | 4-1BB | |
| ratios | Untransduced | Transduced | Untransduced | Transduced |
| 1:1 | 8% | 10% | 8% | 20% |
| 2.5:1 | 8% | 20% | 8% | 25% |
| 5:1 | 10% | 25% | 8% | 45% |
| 10:1 | 10% | 40% | 8% | 60% |

The data shown in FIG. 19 and table 3 suggested that the dual bi-specific CAR constructs possess potent anti-tumor activity against the target cancer cell lines. Notably, the co-expression of either CD20/CD19 or CD22/CD19 enhances the anti-tumor activity in comparison to the conventional second generation CAR T cells. Thus, third generation bi-specific CAR T cells of the present invention having ICOS and 4-1BB co-stimulatory domains are superior and have more potent anti-tumor killing potential than the conventional second generation mono-specific CAR T cells.

### Example 15: CAR T Cell Expansion and Activation Assay

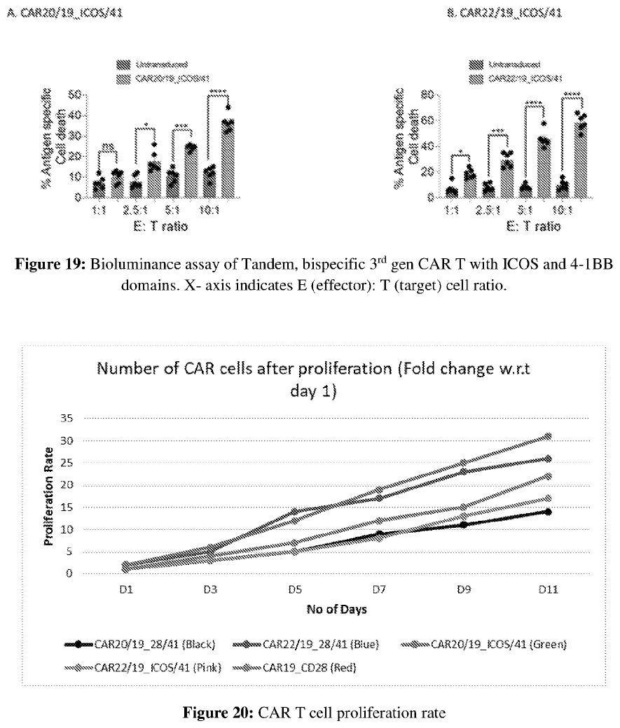

The CAR T cell expansion assay was performed to determine the in vitro persistence of the cells. The expansion assay was performed by first co-culturing the CAR T cells with various ratios of the target Raji cells in 1:10 E:T ratio for various time points. After the initial co-culture (Day1), tumor cell challenge was done at Day 2, 4, 6, 8 and 10. The tumor cells and CAR T cells were harvested at the indicated time points and analyzed by flow cytometry. To distinguish the CAR T cells from Raji cell, the co-culture was stained with anti-CD3 antibody and gating was applied on CD3 positive population. Then, the CAR T cell number was determined at various time points. The percentage of the CD3 positive cells was plotted at different time points, representing the proliferation/expansion rate, which is represented as fold change with respect to the Dayl (D1)) of the CAR T cells as indicated in FIG. 20. The initial CAR T cell number was kept constant for the assay.

TABLE 4
Data showing number of cells of CAR T cells expressing various co-stimulatory
domains for defining the proliferation rate which is represented as fold
change with respect to the Day 1 (D 1) as shown in FIG. 20.

| | Number of CAR cells after profliferation (Fold change w.r.t day 1) | | | | |
|---|---|---|---|---|---|
| | CAR20/19_{—} | CAR22/19_{—} | CAR20/19_{—} | CAR22/19_{—} | CAR19_{—} |
| Days | 28/41 (Black) | 28/41 (Blue) | ICOS/41 (Green) | ICOS/41 (Pink) | CD28 (Red) |
| D 1 | 1 | 2 | 2 | 1 | 1 |
| D 3 | 3 | 5 | 6 | 3 | 4 |
| D 5 | 5 | 14 | 12 | 5 | 7 |
| D 7 | 9 | 17 | 19 | 8 | 12 |
| D 9 | 11 | 23 | 25 | 13 | 15 |
| D 11 | 14 | 26 | 31 | 17 | 22 |

The proliferation assay indicated that the bi-specific CAR T cells such as CD20/CD19 or CD22/CD19 expressing two co-stimulatory domains, exhibit better proliferation rate than the CD19 CAR T cells with a single co-stimulatory domain.

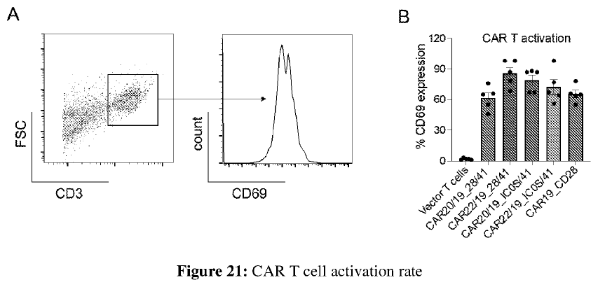

After 24 hours of the co-culture of Raji cells with CAR T cells (1:10), the activation assay was performed by flow cytometry. The co-cultured cells were stained with anti-CD3 and anti-CD69 antibody simultaneously and gating was applied to CD3 population. Then, the percentage of the CD69 cell number was determined which indicated the percentage of CAR T cell activation as provided in FIG. 21.

TABLE 5
Data showing percentage of proliferation rate of CAR T cells expressing
various co-stimulatory domains as shown in FIG. 21.

| CAR T Cell | Percentage of CD69 Expression |
|---|---|
| Vector T cells | 1.8% |
| CAR20/19_28/41 | 61.6% |
| CAR22/19_28/41 | 85.8% |
| CAR20/19_ICOS/41 | 79.2% |
| CAR22/19_ICOS/41 | 72.4% |
| CAR19_CD28 | 65.6% |

The activation assay indicated that the bi-specific CAR T cells such as CD20/CD19 or CD22/CD19 expressing two co-stimulatory domains, exhibited higher activation status than the CD19 CAR T cells with a single co-stimulatory domain.

On the basis of few pilot tests, similar activity or results appear achievable in vivo models or animal studies. The in vivo preclinical animal model is generated with the target Raji cells expressing luciferase reporter and the target antigens like CD19, CD20 and CD22. Currently, various 3^{rd} and 4^{th} generation CAR-T cells are being evaluated in the pre-clinical models with the end points measured as anti-tumor activity. CAR-T cell expansion kinetics, CAR-T cell persistence, CAR-T cell proliferation and pharmacological study to determine any toxicity. More detailed studies are underway.

### ADVANTAGES

1. The CAR constructs of the present invention are next generation chimeric antigen receptor (CAR) platform that functions as a modular system.
2. The present contstructs are indigenous next-generation tandem bispecific CAR constructs that target CD19 and CD20 antigens simultaneously and similarly CD19 and CD22 antigens.
3. The present invention also provides a multi-antigen system that include all three antigens (CD19, CD20 and CD22).
4. The present invention mitigates target antigen escape/loss and down-regulation as a mechanism of relapse and overcome the clinical challenges of tumour heterogeneity while maintaining significant antitumor potency.
5. The robust CAR T cell function achieves enhanced persistence and T cell function by integrating signalling domains from one or more T cell co-stimulatory signaling molecules, selected from ICOS, 41BB, OX40, CD27 and CD28 into the synthetic CAR.
6. The CAR constructs of the present invention are efficient and were generated by positioning intracellular costimulatory domains within tandem bispecific CARs by a combinatorial approach.

1. A bispecific chimeric antigen receptor (CAR) comprising:
(a) a CD19 binding domain along with CD20 or CD22;
(b) a linker region;
(c) a hinge or spacer region;
(d) a transmembrane region;
(e) an intracellular signaling domain; and
(f) two or more intracellular co-stimulatory signalling domains selected from the group consisting of CD28, 41BB, OX40 and ICOS.

2. The chimeric antigen receptor (CAR) as claimed in claim 1, comprising an amino acid sequence of CD19, CD20 and CD22 binding domains as set forth in SEQ ID NOs: 21, 22 and 23 respectively.

3. The chimeric antigen receptor (CAR) as claimed in claim 1, wherein the linker region is selected from the group consisting of (G2S)₃-linker; (G3S)₃-linker, (G4S)₃-linker, (G2S)₄-linker, (G3S)₄-linker, (G4S)₄-linker, (G2S)₅-linker, (G3S)₅-linker, or (G4S)₅-linker.

4. The chimeric antigen receptor (CAR) as claimed in claim 1, wherein the hinge or spacer region is CD8 having an amino acid sequence as set forth in SEQ ID NO: 25.

5. The chimeric antigen receptor (CAR) as claimed in claim 1, wherein the transmembrane region is CD8 or ICOS having an amino acid sequence as set forth in SEQ ID NOs: 26 and 27 respectively.

6. The chimeric antigen receptor (CAR) as claimed in claim 1, wherein the intracellular signaling domain is human CD3ζ having an amino acid sequence as set forth in SEQ ID NO: 31.

7. The chimeric antigen receptor (CAR) as claimed in claim 1, wherein the linker region comprises an amino acid sequence as set forth in SEQ ID NO: 24.

8. The chimeric antigen receptor (CAR) as claimed in claim 1, wherein co-stimulatory signaling domains ICOS, OX40, 41BB and CD28 comprise an amino acid sequence as set forth in SEQ ID NOs: 28, 29, 30 and 31 respectively.

9. The chimeric antigen receptor (CAR) as claimed in claims 1-8, comprising an amino acid sequence as set forth in SEQ ID NOs: 11-20.

10. A bispecific chimeric antigen receptor (CAR) comprising:
i. a CD20 binding domain followed by CD19 scFv bispecific ScFv in tandem separated by the GS linker;
ii. a combination of co-stimulatory domains selected from CD28 and 41BB, OX40 and 41BB, ICOS and OX40, ICOS and 41BB, and ICOS, OX40 and 41BB; and
iii. a CD3 zeta signalling domain.

11. A bispecific chimeric antigen receptor (CAR) comprising:
i. a CD22 binding domain followed by CD19 scFv bispecific ScFv in tandem separated by the GS linker;
ii. a combination of co-stimulatory domains selected from CD28 and 41BB, OX40 and 41BB, ICOS and OX40, ICOS and 41BB, and ICOS, OX40 and 41BB; and
iii. a CD3 zeta signalling domain.

12. A polynucleotide encoding a CAR as claimed in claims 1 to 11.

13. A vector comprising the polynucleotide as claimed in claim 12.

14. The vector as claimed in claim 13, wherein the vector is an expression vector selected from the group consisting of lentiviral or retroviral vectors.

15. The vector as claimed in claim 13 or 14, comprising CMV promoter/enhancer, a Psi (Ψ) packaging signal, a central polypurine tract/central termination sequence (cPPT/CTS), an EF1 alpha promoter for optimal expression in T cells, a linker/hinge linked to the binding domain, woodchuck hepatitis virus posttranscriptional regulatory element (WPRE), f1 ori and promoter for ampicillin resistance.

16. A composition comprising the CAR as claimed in claims 1-11 or the vector comprising the polynucleotide encoding the CAR as claimed in claims 12-15 and a pharmaceutically acceptable excipient.

17. A method of treating a B cell related condition in a subject in need thereof, comprising administering to the subject a therapeutically effect amount of the composition of claim 16.

18. The method of claim 17, wherein the B cell related condition is Acute Lymphoblastic Leukemia, B cell lymphomas, multiple myeloma, non-Hodgkin's lymphoma, B cell proliferations of uncertain malignant potential, lymphomatoid granulomatosis, post-transplant lymphoproliferative disorder, mantle cell lymphoma, follicular lymphoma, or Burkitt lymphoma.

19. The method of claim 17, wherein the B cell related condition is a B cell malignancy.

20. The method of claim 19, wherein the B cell malignancy is Acute Lymphoblastic Leukemia and B cell lymphomas.

21. The CAR as claimed in claims 1-11 or the polynucleotide encoding a CAR as claimed in claim 12, for use in treating B-cell malignancy.


SEQUENCE LISTING (32 sequences)

SEQ ID NO: 1 - DNA, 3557 bp, synthetic construct
  source 1..3557: mol_type = other DNA
ggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaaccgccatggcgctccctgtcaccgcactgcttcttccgctggcactgctgctgcacgctgcacggcctgaggtgcagctgcagcagagcggcgccgagctggtgaagcccggcgccagcgtgaagatgagctgcaaggccagcggctacaccttcttcagctacaacatgcactgggtgaagcagacccccggccagggcctggagtggatcggcgccatctaccccggcaacggcgacaccagctacaaccagaagttcaagggcaaggccaccctgaccgccgacaagagcagcagcaccgcctacatgcagctgagcagcctgaccagcgaggacagcgccgactactactgcgccaggagcaactactacggcagcagctactggttcttcgacgtgtggggcgccggcaccaccgtgaccgtgagcagcggcggcggcggcagcggcggcggcggcagcggcggcggcggcagcgacatcgtgctgacccagagccccgccatcctgagcgccagccccggcgagaaggtgaccatgacctgcagggccagcagcagcgtgaactacatggactggtatcagaagaagcccggcagcagccccaagccctggatctacgccaccagcaacctggccagcggcgtgcccgccaggttcagcggcagcggcagcggcaccagctacagcctgaccatcagcagggtggaggccgaggacgccgccacctactactgccagcagtggagcttcaacccccccaccttcggcggcggcaccaagctggagatcaagggcggcggcggcagcggcggcggcggcagcggcggcggcggcagcggcggcggcggcagcggcggcggcggcagcgatatacagatgacgcagacaacgtcaagtctttccgccagcttgggagaccgagtgactatatcttgtagagcaagccaggatatttctaagtatcttaactggtaccaacaaaagcccgatggaacggttaagctgcttatataccataccagtagactccactccggcgtaccatcacggttttctggcagtggctccgggaccgactattctttgacgatctctaatctcgaacaagaggatattgcaacatacttttgtcagcaaggcaataccttgccatatacgtttgggggcgggacaaaacttgagataaccggcggcggtggttcaggcggtggcggttccggtggtgggggatcagaggttaagcttcaggaatccggaccaggtttggttgcccccagccaatctctcagcgttacatgcacggtttcaggcgtcagtctccccgattacggtgtaagttggattcggcaacctccgcgaaagggtctggaatggctgggggttatttgggggagtgagacaacttattacaactctgcacttaagagtcggcttaccatcatcaaggataattcaaaatcacaagtattcctgaagatgaactcattgcaaacagatgatacagctatatactattgtgccaagcattactattatggtggttcttatgcaatggattactgggggcaaggcacgtcagtgacagtgagttcaaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaggagggaccagaggctgccccccgacgcccacaagccccccggcggcggcagcttcaggacccccatccaggaggagcaggccgacgcccacagcaccctggccaagatcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcg

SEQ ID NO: 2 - DNA, 3569 bp, synthetic construct
  source 1..3569: mol_type = other DNA
ggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaaccgccatggcgctccctgtcaccgcactgcttcttccgctggcactgctgctgcacgctgcacggcctgaggtgcagctgcagcagagcggcgccgagctggtgaagcccggcgccagcgtgaagatgagctgcaaggccagcggctacaccttcttcagctacaacatgcactgggtgaagcagacccccggccagggcctggagtggatcggcgccatctaccccggcaacggcgacaccagctacaaccagaagttcaagggcaaggccaccctgaccgccgacaagagcagcagcaccgcctacatgcagctgagcagcctgaccagcgaggacagcgccgactactactgcgccaggagcaactactacggcagcagctactggttcttcgacgtgtggggcgccggcaccaccgtgaccgtgagcagcggcggcggcggcagcggcggcggcggcagcggcggcggcggcagcgacatcgtgctgacccagagccccgccatcctgagcgccagccccggcgagaaggtgaccatgacctgcagggccagcagcagcgtgaactacatggactggtatcagaagaagcccggcagcagccccaagccctggatctacgccaccagcaacctggccagcggcgtgcccgccaggttcagcggcagcggcagcggcaccagctacagcctgaccatcagcagggtggaggccgaggacgccgccacctactactgccagcagtggagcttcaacccccccaccttcggcggcggcaccaagctggagatcaagggcggcggcggcagcggcggcggcggcagcggcggcggcggcagcggcggcggcggcagcggcggcggcggcagcgatatacagatgacgcagacaacgtcaagtctttccgccagcttgggagaccgagtgactatatcttgtagagcaagccaggatatttctaagtatcttaactggtaccaacaaaagcccgatggaacggttaagctgcttatataccataccagtagactccactccggcgtaccatcacggttttctggcagtggctccgggaccgactattctttgacgatctctaatctcgaacaagaggatattgcaacatacttttgtcagcaaggcaataccttgccatatacgtttgggggcgggacaaaacttgagataaccggcggcggtggttcaggcggtggcggttccggtggtgggggatcagaggttaagcttcaggaatccggaccaggtttggttgcccccagccaatctctcagcgttacatgcacggtttcaggcgtcagtctccccgattacggtgtaagttggattcggcaacctccgcgaaagggtctggaatggctgggggttatttgggggagtgagacaacttattacaactctgcacttaagagtcggcttaccatcatcaaggataattcaaaatcacaagtattcctgaagatgaactcattgcaaacagatgatacagctatatactattgtgccaagcattactattatggtggttcttatgcaatggattactgggggcaaggcacgtcagtgacagtgagttcaaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaggagcaagaggagcaggctgctgcacagcgactacatgaacatgacccccaggaggcccggccccaccaggaagcactaccagccctacgccccccccagggacttcgccgcctacaggagcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcg

SEQ ID NO: 3 - DNA, 3662 bp, synthetic construct
  source 1..3662: mol_type = other DNA
ggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaaccgccatggcgctccctgtcaccgcactgcttcttccgctggcactgctgctgcacgctgcacggcctgaggtgcagctgcagcagagcggcgccgagctggtgaagcccggcgccagcgtgaagatgagctgcaaggccagcggctacaccttcttcagctacaacatgcactgggtgaagcagacccccggccagggcctggagtggatcggcgccatctaccccggcaacggcgacaccagctacaaccagaagttcaagggcaaggccaccctgaccgccgacaagagcagcagcaccgcctacatgcagctgagcagcctgaccagcgaggacagcgccgactactactgcgccaggagcaactactacggcagcagctactggttcttcgacgtgtggggcgccggcaccaccgtgaccgtgagcagcggcggcggcggcagcggcggcggcggcagcggcggcggcggcagcgacatcgtgctgacccagagccccgccatcctgagcgccagccccggcgagaaggtgaccatgacctgcagggccagcagcagcgtgaactacatggactggtatcagaagaagcccggcagcagccccaagccctggatctacgccaccagcaacctggccagcggcgtgcccgccaggttcagcggcagcggcagcggcaccagctacagcctgaccatcagcagggtggaggccgaggacgccgccacctactactgccagcagtggagcttcaacccccccaccttcggcggcggcaccaagctggagatcaagggcggcggcggcagcggcggcggcggcagcggcggcggcggcagcggcggcggcggcagcggcggcggcggcagcgatatacagatgacgcagacaacgtcaagtctttccgccagcttgggagaccgagtgactatatcttgtagagcaagccaggatatttctaagtatcttaactggtaccaacaaaagcccgatggaacggttaagctgcttatataccataccagtagactccactccggcgtaccatcacggttttctggcagtggctccgggaccgactattctttgacgatctctaatctcgaacaagaggatattgcaacatacttttgtcagcaaggcaataccttgccatatacgtttgggggcgggacaaaacttgagataaccggcggcggtggttcaggcggtggcggttccggtggtgggggatcagaggttaagcttcaggaatccggaccaggtttggttgcccccagccaatctctcagcgttacatgcacggtttcaggcgtcagtctccccgattacggtgtaagttggattcggcaacctccgcgaaagggtctggaatggctgggggttatttgggggagtgagacaacttattacaactctgcacttaagagtcggcttaccatcatcaaggataattcaaaatcacaagtattcctgaagatgaactcattgcaaacagatgatacagctatatactattgtgccaagcattactattatggtggttcttatgcaatggattactgggggcaaggcacgtcagtgacagtgagttcaaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatttctggctgcccatcggctgcgccgccttcgtggtggtgtgcatcctgggctgcatcctgatctgctggctgaccaagaagaagtacagcagcagcgtgcacgaccccaacggcgagtacatgttcatgagggccgtgaacaccgccaagaagagcaggctgaccgacgtgaccctgaggagggaccagaggctgccccccgacgcccacaagccccccggcggcggcagcttcaggacccccatccaggaggagcaggccgacgcccacagcaccctggccaagatcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcg

SEQ ID NO: 4 - DNA, 3572 bp, synthetic construct
  source 1..3572: mol_type = other DNA
ggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaaccgccatggcgctccctgtcaccgcactgcttcttccgctggcactgctgctgcacgctgcacggcctcaggtgcagctgcagcagagcggccccggcctggtgaagcccagccagaccctgagcctgacctgcgccatcagcggcgacagcgtgagcagcaacagcgccgcctggaactggatcaggcagagccccagcaggggcctggagtggctgggcaggacctactacaggagcaagtggtacaacgactacgccgtgagcgtgaagagcaggatcaccatcaaccccgacaccagcaagaaccagttcagcctgcagctgaacagcgtgacccccgaggacaccgccgtgtactactgcgccagggaggtgaccggcgacctggaggacgccttcgacatctggggccagggcaccatggtgaccgtgagcagcggcggcggcggcagcggcggcggcggcagcggcggcggcggcagcgacatccagatgacccagagccccagcagcctgagcgccagcgtgggcgacagggtgaccatcacctgcagggccagccagaccatctggagctacctgaactggtatcagcagaggcccggcaaggcccccaacctgctgatctacgccgccagcagcctgcagagcggcgtgcccagcaggttcagcggcaggggcagcggcaccgacttcaccctgaccatcagcagcctgcaggccgaggacttcgccacctactactgccagcagagctacagcatcccccagaccttcggccagggcaccaagctggagatcaagaggaccggcggcggcggcagcggcggcggcggcagcggcggcggcggcagcggcggcggcggcagcggcggcggcggcagcgatatacagatgacgcagacaacgtcaagtctttccgccagcttgggagaccgagtgactatatcttgtagagcaagccaggatatttctaagtatcttaactggtaccaacaaaagcccgatggaacggttaagctgcttatataccataccagtagactccactccggcgtaccatcacggttttctggcagtggctccgggaccgactattctttgacgatctctaatctcgaacaagaggatattgcaacatacttttgtcagcaaggcaataccttgccatatacgtttgggggcgggacaaaacttgagataaccggcggcggtggttcaggcggtggcggttccggtggtgggggatcagaggttaagcttcaggaatccggaccaggtttggttgcccccagccaatctctcagcgttacatgcacggtttcaggcgtcagtctccccgattacggtgtaagttggattcggcaacctccgcgaaagggtctggaatggctgggggttatttgggggagtgagacaacttattacaactctgcacttaagagtcggcttaccatcatcaaggataattcaaaatcacaagtattcctgaagatgaactcattgcaaacagatgatacagctatatactattgtgccaagcattactattatggtggttcttatgcaatggattactgggggcaaggcacgtcagtgacagtgagttcaaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaggagggaccagaggctgccccccgacgcccacaagccccccggcggcggcagcttcaggacccccatccaggaggagcaggccgacgcccacagcaccctggccaagatcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcg

SEQ ID NO: 5 - DNA, 3677 bp, synthetic construct
  source 1..3677: mol_type = other DNA
ggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaaccgccatggcgctccctgtcaccgcactgcttcttccgctggcactgctgctgcacgctgcacggcctcaggtgcagctgcagcagagcggccccggcctggtgaagcccagccagaccctgagcctgacctgcgccatcagcggcgacagcgtgagcagcaacagcgccgcctggaactggatcaggcagagccccagcaggggcctggagtggctgggcaggacctactacaggagcaagtggtacaacgactacgccgtgagcgtgaagagcaggatcaccatcaaccccgacaccagcaagaaccagttcagcctgcagctgaacagcgtgacccccgaggacaccgccgtgtactactgcgccagggaggtgaccggcgacctggaggacgccttcgacatctggggccagggcaccatggtgaccgtgagcagcggcggcggcggcagcggcggcggcggcagcggcggcggcggcagcgacatccagatgacccagagccccagcagcctgagcgccagcgtgggcgacagggtgaccatcacctgcagggccagccagaccatctggagctacctgaactggtatcagcagaggcccggcaaggcccccaacctgctgatctacgccgccagcagcctgcagagcggcgtgcccagcaggttcagcggcaggggcagcggcaccgacttcaccctgaccatcagcagcctgcaggccgaggacttcgccacctactactgccagcagagctacagcatcccccagaccttcggccagggcaccaagctggagatcaagaggaccggcggcggcggcagcggcggcggcggcagcggcggcggcggcagcggcggcggcggcagcggcggcggcggcagcgatatacagatgacgcagacaacgtcaagtctttccgccagcttgggagaccgagtgactatatcttgtagagcaagccaggatatttctaagtatcttaactggtaccaacaaaagcccgatggaacggttaagctgcttatataccataccagtagactccactccggcgtaccatcacggttttctggcagtggctccgggaccgactattctttgacgatctctaatctcgaacaagaggatattgcaacatacttttgtcagcaaggcaataccttgccatatacgtttgggggcgggacaaaacttgagataaccggcggcggtggttcaggcggtggcggttccggtggtgggggatcagaggttaagcttcaggaatccggaccaggtttggttgcccccagccaatctctcagcgttacatgcacggtttcaggcgtcagtctccccgattacggtgtaagttggattcggcaacctccgcgaaagggtctggaatggctgggggttatttgggggagtgagacaacttattacaactctgcacttaagagtcggcttaccatcatcaaggataattcaaaatcacaagtattcctgaagatgaactcattgcaaacagatgatacagctatatactattgtgccaagcattactattatggtggttcttatgcaatggattactgggggcaaggcacgtcagtgacagtgagttcaaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatttctggctgcccatcggctgcgccgccttcgtggtggtgtgcatcctgggctgcatcctgatctgctggctgaccaagaagaagtacagcagcagcgtgcacgaccccaacggcgagtacatgttcatgagggccgtgaacaccgccaagaagagcaggctgaccgacgtgaccctgaggagggaccagaggctgccccccgacgcccacaagccccccggcggcggcagcttcaggacccccatccaggaggagcaggccgacgcccacagcaccctggccaagatcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcg

SEQ ID NO: 6 - DNA, 3584 bp, synthetic construct
  source 1..3584: mol_type = other DNA
ggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaaccgccatggcgctccctgtcaccgcactgcttcttccgctggcactgctgctgcacgctgcacggcctcaggtgcagctgcagcagagcggccccggcctggtgaagcccagccagaccctgagcctgacctgcgccatcagcggcgacagcgtgagcagcaacagcgccgcctggaactggatcaggcagagccccagcaggggcctggagtggctgggcaggacctactacaggagcaagtggtacaacgactacgccgtgagcgtgaagagcaggatcaccatcaaccccgacaccagcaagaaccagttcagcctgcagctgaacagcgtgacccccgaggacaccgccgtgtactactgcgccagggaggtgaccggcgacctggaggacgccttcgacatctggggccagggcaccatggtgaccgtgagcagcggcggcggcggcagcggcggcggcggcagcggcggcggcggcagcgacatccagatgacccagagccccagcagcctgagcgccagcgtgggcgacagggtgaccatcacctgcagggccagccagaccatctggagctacctgaactggtatcagcagaggcccggcaaggcccccaacctgctgatctacgccgccagcagcctgcagagcggcgtgcccagcaggttcagcggcaggggcagcggcaccgacttcaccctgaccatcagcagcctgcaggccgaggacttcgccacctactactgccagcagagctacagcatcccccagaccttcggccagggcaccaagctggagatcaagaggaccggcggcggcggcagcggcggcggcggcagcggcggcggcggcagcggcggcggcggcagcggcggcggcggcagcgatatacagatgacgcagacaacgtcaagtctttccgccagcttgggagaccgagtgactatatcttgtagagcaagccaggatatttctaagtatcttaactggtaccaacaaaagcccgatggaacggttaagctgcttatataccataccagtagactccactccggcgtaccatcacggttttctggcagtggctccgggaccgactattctttgacgatctctaatctcgaacaagaggatattgcaacatacttttgtcagcaaggcaataccttgccatatacgtttgggggcgggacaaaacttgagataaccggcggcggtggttcaggcggtggcggttccggtggtgggggatcagaggttaagcttcaggaatccggaccaggtttggttgcccccagccaatctctcagcgttacatgcacggtttcaggcgtcagtctccccgattacggtgtaagttggattcggcaacctccgcgaaagggtctggaatggctgggggttatttgggggagtgagacaacttattacaactctgcacttaagagtcggcttaccatcatcaaggataattcaaaatcacaagtattcctgaagatgaactcattgcaaacagatgatacagctatatactattgtgccaagcattactattatggtggttcttatgcaatggattactgggggcaaggcacgtcagtgacagtgagttcaaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaggagcaagaggagcaggctgctgcacagcgactacatgaacatgacccccaggaggcccggccccaccaggaagcactaccagccctacgccccccccagggacttcgccgcctacaggagcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcg

SEQ ID NO: 7 - DNA, 3536 bp, synthetic construct
  source 1..3536: mol_type = other DNA
ggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaaccgccatggcgctccctgtcaccgcactgcttcttccgctggcactgctgctgcacgctgcacggcctgaggtgcagctgcagcagagcggcgccgagctggtgaagcccggcgccagcgtgaagatgagctgcaaggccagcggctacaccttcttcagctacaacatgcactgggtgaagcagacccccggccagggcctggagtggatcggcgccatctaccccggcaacggcgacaccagctacaaccagaagttcaagggcaaggccaccctgaccgccgacaagagcagcagcaccgcctacatgcagctgagcagcctgaccagcgaggacagcgccgactactactgcgccaggagcaactactacggcagcagctactggttcttcgacgtgtggggcgccggcaccaccgtgaccgtgagcagcggcggcggcggcagcggcggcggcggcagcggcggcggcggcagcgacatcgtgctgacccagagccccgccatcctgagcgccagccccggcgagaaggtgaccatgacctgcagggccagcagcagcgtgaactacatggactggtatcagaagaagcccggcagcagccccaagccctggatctacgccaccagcaacctggccagcggcgtgcccgccaggttcagcggcagcggcagcggcaccagctacagcctgaccatcagcagggtggaggccgaggacgccgccacctactactgccagcagtggagcttcaacccccccaccttcggcggcggcaccaagctggagatcaagggcggcggcggcagcggcggcggcggcagcggcggcggcggcagcggcggcggcggcagcggcggcggcggcagcgatatacagatgacgcagacaacgtcaagtctttccgccagcttgggagaccgagtgactatatcttgtagagcaagccaggatatttctaagtatcttaactggtaccaacaaaagcccgatggaacggttaagctgcttatataccataccagtagactccactccggcgtaccatcacggttttctggcagtggctccgggaccgactattctttgacgatctctaatctcgaacaagaggatattgcaacatacttttgtcagcaaggcaataccttgccatatacgtttgggggcgggacaaaacttgagataaccggcggcggtggttcaggcggtggcggttccggtggtgggggatcagaggttaagcttcaggaatccggaccaggtttggttgcccccagccaatctctcagcgttacatgcacggtttcaggcgtcagtctccccgattacggtgtaagttggattcggcaacctccgcgaaagggtctggaatggctgggggttatttgggggagtgagacaacttattacaactctgcacttaagagtcggcttaccatcatcaaggataattcaaaatcacaagtattcctgaagatgaactcattgcaaacagatgatacagctatatactattgtgccaagcattactattatggtggttcttatgcaatggattactgggggcaaggcacgtcagtgacagtgagttcaaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatttctggctgcccatcggctgcgccgccttcgtggtggtgtgcatcctgggctgcatcctgatctgctggctgaccaagaagaagtacagcagcagcgtgcacgaccccaacggcgagtacatgttcatgagggccgtgaacaccgccaagaagagcaggctgaccgacgtgaccctgaggagggaccagaggctgccccccgacgcccacaagccccccggcggcggcagcttcaggacccccatccaggaggagcaggccgacgcccacagcaccctggccaagatcagagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcg

SEQ ID NO: 8 - DNA, 3551 bp, synthetic construct
  source 1..3551: mol_type = other DNA
ggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaaccgccatggcgctccctgtcaccgcactgcttcttccgctggcactgctgctgcacgctgcacggcctcaggtgcagctgcagcagagcggccccggcctggtgaagcccagccagaccctgagcctgacctgcgccatcagcggcgacagcgtgagcagcaacagcgccgcctggaactggatcaggcagagccccagcaggggcctggagtggctgggcaggacctactacaggagcaagtggtacaacgactacgccgtgagcgtgaagagcaggatcaccatcaaccccgacaccagcaagaaccagttcagcctgcagctgaacagcgtgacccccgaggacaccgccgtgtactactgcgccagggaggtgaccggcgacctggaggacgccttcgacatctggggccagggcaccatggtgaccgtgagcagcggcggcggcggcagcggcggcggcggcagcggcggcggcggcagcgacatccagatgacccagagccccagcagcctgagcgccagcgtgggcgacagggtgaccatcacctgcagggccagccagaccatctggagctacctgaactggtatcagcagaggcccggcaaggcccccaacctgctgatctacgccgccagcagcctgcagagcggcgtgcccagcaggttcagcggcaggggcagcggcaccgacttcaccctgaccatcagcagcctgcaggccgaggacttcgccacctactactgccagcagagctacagcatcccccagaccttcggccagggcaccaagctggagatcaagaggaccggcggcggcggcagcggcggcggcggcagcggcggcggcggcagcggcggcggcggcagcggcggcggcggcagcgatatacagatgacgcagacaacgtcaagtctttccgccagcttgggagaccgagtgactatatcttgtagagcaagccaggatatttctaagtatcttaactggtaccaacaaaagcccgatggaacggttaagctgcttatataccataccagtagactccactccggcgtaccatcacggttttctggcagtggctccgggaccgactattctttgacgatctctaatctcgaacaagaggatattgcaacatacttttgtcagcaaggcaataccttgccatatacgtttgggggcgggacaaaacttgagataaccggcggcggtggttcaggcggtggcggttccggtggtgggggatcagaggttaagcttcaggaatccggaccaggtttggttgcccccagccaatctctcagcgttacatgcacggtttcaggcgtcagtctccccgattacggtgtaagttggattcggcaacctccgcgaaagggtctggaatggctgggggttatttgggggagtgagacaacttattacaactctgcacttaagagtcggcttaccatcatcaaggataattcaaaatcacaagtattcctgaagatgaactcattgcaaacagatgatacagctatatactattgtgccaagcattactattatggtggttcttatgcaatggattactgggggcaaggcacgtcagtgacagtgagttcaaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatttctggctgcccatcggctgcgccgccttcgtggtggtgtgcatcctgggctgcatcctgatctgctggctgaccaagaagaagtacagcagcagcgtgcacgaccccaacggcgagtacatgttcatgagggccgtgaacaccgccaagaagagcaggctgaccgacgtgaccctgaggagggaccagaggctgccccccgacgcccacaagccccccggcggcggcagcttcaggacccccatccaggaggagcaggccgacgcccacagcaccctggccaagatcagagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcg

SEQ ID NO: 9 - DNA, 3551 bp, synthetic construct
  source 1..3551: mol_type = other DNA
ggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaaccgccatggcgctccctgtcaccgcactgcttcttccgctggcactgctgctgcacgctgcacggcctgaggtgcagctgcagcagagcggcgccgagctggtgaagcccggcgccagcgtgaagatgagctgcaaggccagcggctacaccttcttcagctacaacatgcactgggtgaagcagacccccggccagggcctggagtggatcggcgccatctaccccggcaacggcgacaccagctacaaccagaagttcaagggcaaggccaccctgaccgccgacaagagcagcagcaccgcctacatgcagctgagcagcctgaccagcgaggacagcgccgactactactgcgccaggagcaactactacggcagcagctactggttcttcgacgtgtggggcgccggcaccaccgtgaccgtgagcagcggcggcggcggcagcggcggcggcggcagcggcggcggcggcagcgacatcgtgctgacccagagccccgccatcctgagcgccagccccggcgagaaggtgaccatgacctgcagggccagcagcagcgtgaactacatggactggtatcagaagaagcccggcagcagccccaagccctggatctacgccaccagcaacctggccagcggcgtgcccgccaggttcagcggcagcggcagcggcaccagctacagcctgaccatcagcagggtggaggccgaggacgccgccacctactactgccagcagtggagcttcaacccccccaccttcggcggcggcaccaagctggagatcaagggcggcggcggcagcggcggcggcggcagcggcggcggcggcagcggcggcggcggcagcggcggcggcggcagcgatatacagatgacgcagacaacgtcaagtctttccgccagcttgggagaccgagtgactatatcttgtagagcaagccaggatatttctaagtatcttaactggtaccaacaaaagcccgatggaacggttaagctgcttatataccataccagtagactccactccggcgtaccatcacggttttctggcagtggctccgggaccgactattctttgacgatctctaatctcgaacaagaggatattgcaacatacttttgtcagcaaggcaataccttgccatatacgtttgggggcgggacaaaacttgagataaccggcggcggtggttcaggcggtggcggttccggtggtgggggatcagaggttaagcttcaggaatccggaccaggtttggttgcccccagccaatctctcagcgttacatgcacggtttcaggcgtcagtctccccgattacggtgtaagttggattcggcaacctccgcgaaagggtctggaatggctgggggttatttgggggagtgagacaacttattacaactctgcacttaagagtcggcttaccatcatcaaggataattcaaaatcacaagtattcctgaagatgaactcattgcaaacagatgatacagctatatactattgtgccaagcattactattatggtggttcttatgcaatggattactgggggcaaggcacgtcagtgacagtgagttcaaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatttctggctgcccatcggctgcgccgccttcgtggtggtgtgcatcctgggctgcatcctgatctgctggctgaccaagaagaagtacagcagcagcgtgcacgaccccaacggcgagtacatgttcatgagggccgtgaacaccgccaagaagagcaggctgaccgacgtgaccctgaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcg

SEQ ID NO: 10 - DNA, 3566 bp, synthetic construct
  source 1..3566: mol_type = other DNA
ggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaaccgccatggcgctccctgtcaccgcactgcttcttccgctggcactgctgctgcacgctgcacggcctcaggtgcagctgcagcagagcggccccggcctggtgaagcccagccagaccctgagcctgacctgcgccatcagcggcgacagcgtgagcagcaacagcgccgcctggaactggatcaggcagagccccagcaggggcctggagtggctgggcaggacctactacaggagcaagtggtacaacgactacgccgtgagcgtgaagagcaggatcaccatcaaccccgacaccagcaagaaccagttcagcctgcagctgaacagcgtgacccccgaggacaccgccgtgtactactgcgccagggaggtgaccggcgacctggaggacgccttcgacatctggggccagggcaccatggtgaccgtgagcagcggcggcggcggcagcggcggcggcggcagcggcggcggcggcagcgacatccagatgacccagagccccagcagcctgagcgccagcgtgggcgacagggtgaccatcacctgcagggccagccagaccatctggagctacctgaactggtatcagcagaggcccggcaaggcccccaacctgctgatctacgccgccagcagcctgcagagcggcgtgcccagcaggttcagcggcaggggcagcggcaccgacttcaccctgaccatcagcagcctgcaggccgaggacttcgccacctactactgccagcagagctacagcatcccccagaccttcggccagggcaccaagctggagatcaagaggaccggcggcggcggcagcggcggcggcggcagcggcggcggcggcagcggcggcggcggcagcggcggcggcggcagcgatatacagatgacgcagacaacgtcaagtctttccgccagcttgggagaccgagtgactatatcttgtagagcaagccaggatatttctaagtatcttaactggtaccaacaaaagcccgatggaacggttaagctgcttatataccataccagtagactccactccggcgtaccatcacggttttctggcagtggctccgggaccgactattctttgacgatctctaatctcgaacaagaggatattgcaacatacttttgtcagcaaggcaataccttgccatatacgtttgggggcgggacaaaacttgagataaccggcggcggtggttcaggcggtggcggttccggtggtgggggatcagaggttaagcttcaggaatccggaccaggtttggttgcccccagccaatctctcagcgttacatgcacggtttcaggcgtcagtctccccgattacggtgtaagttggattcggcaacctccgcgaaagggtctggaatggctgggggttatttgggggagtgagacaacttattacaactctgcacttaagagtcggcttaccatcatcaaggataattcaaaatcacaagtattcctgaagatgaactcattgcaaacagatgatacagctatatactattgtgccaagcattactattatggtggttcttatgcaatggattactgggggcaaggcacgtcagtgacagtgagttcaaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatttctggctgcccatcggctgcgccgccttcgtggtggtgtgcatcctgggctgcatcctgatctgctggctgaccaagaagaagtacagcagcagcgtgcacgaccccaacggcgagtacatgttcatgagggccgtgaacaccgccaagaagagcaggctgaccgacgtgaccctgaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcg

SEQ ID NO: 11 - protein, 791 aa, synthetic construct
MALPVTALLLPLALLLHAARPEVQLQQSGAELVKPGASVKMSCKASGYTFFSYNMHWVKQTPGQGLEWIGAIYPGNGDTSYNQKFKGKATLTADKSSSTAYMQLSSLTSEDSADYYCARSNYYGSSYWFFDVWGAGTTVTVSSGGGGSGGGGSGGGGSDIVLTQSPAILSASPGEKVTMTCRASSSVNYMDWYQKKPGSSPKPWIYATSNLASGVPARFSGSGSGTSYSLTISRVEAEDAATYYCQQWSFNPPTFGGGTKLEIKGGGGSGGGGSGGGGSGGGGSGGGGSDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLYCRRDQRLPPDAHKPPGGGSFRTPIQEEQADAHSTLAKIKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 12 - protein, 795 aa, synthetic construct
MALPVTALLLPLALLLHAARPEVQLQQSGAELVKPGASVKMSCKASGYTFFSYNMHWVKQTPGQGLEWIGAIYPGNGDTSYNQKFKGKATLTADKSSSTAYMQLSSLTSEDSADYYCARSNYYGSSYWFFDVWGAGTTVTVSSGGGGSGGGGSGGGGSDIVLTQSPAILSASPGEKVTMTCRASSSVNYMDWYQKKPGSSPKPWIYATSNLASGVPARFSGSGSGTSYSLTISRVEAEDAATYYCQQWSFNPPTFGGGTKLEIKGGGGSGGGGSGGGGSGGGGSGGGGSDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLYCRSKRSRLLHSDYMNMTPRRPGPTRKHYQPYAPPRDFAAYRSKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 13 - protein, 826 aa, synthetic construct
MALPVTALLLPLALLLHAARPEVQLQQSGAELVKPGASVKMSCKASGYTFFSYNMHWVKQTPGQGLEWIGAIYPGNGDTSYNQKFKGKATLTADKSSSTAYMQLSSLTSEDSADYYCARSNYYGSSYWFFDVWGAGTTVTVSSGGGGSGGGGSGGGGSDIVLTQSPAILSASPGEKVTMTCRASSSVNYMDWYQKKPGSSPKPWIYATSNLASGVPARFSGSGSGTSYSLTISRVEAEDAATYYCQQWSFNPPTFGGGTKLEIKGGGGSGGGGSGGGGSGGGGSGGGGSDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACRPAAGGAVHTRGLDFACDFWLPIGCAAFVVVCILGCILICWLTKKKYSSSVHDPNGEYMFMRAVNTAKKSRLTDVTLRRDQRLPPDAHKPPGGGSFRTPIQEEQADAHSTLAKIKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 14 - protein, 796 aa, synthetic construct
MALPVTALLLPLALLLHAARPQVQLQQSGPGLVKPSQTLSLTCAISGDSVSSNSAAWNWIRQSPSRGLEWLGRTYYRSKWYNDYAVSVKSRITINPDTSKNQFSLQLNSVTPEDTAVYYCAREVTGDLEDAFDIWGQGTMVTVSSGGGGSGGGGSGGGGSDIQMTQSPSSLSASVGDRVTITCRASQTIWSYLNWYQQRPGKAPNLLIYAASSLQSGVPSRFSGRGSGTDFTLTISSLQAEDFATYYCQQSYSIPQTFGQGTKLEIKRTGGGGSGGGGSGGGGSGGGGSGGGGSDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLYCRRDQRLPPDAHKPPGGGSFRTPIQEEQADAHSTLAKIKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 15 - protein, 831 aa, synthetic construct
MALPVTALLLPLALLLHAARPQVQLQQSGPGLVKPSQTLSLTCAISGDSVSSNSAAWNWIRQSPSRGLEWLGRTYYRSKWYNDYAVSVKSRITINPDTSKNQFSLQLNSVTPEDTAVYYCAREVTGDLEDAFDIWGQGTMVTVSSGGGGSGGGGSGGGGSDIQMTQSPSSLSASVGDRVTITCRASQTIWSYLNWYQQRPGKAPNLLIYAASSLQSGVPSRFSGRGSGTDFTLTISSLQAEDFATYYCQQSYSIPQTFGQGTKLEIKRTGGGGSGGGGSGGGGSGGGGSGGGGSDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACRPAAGGAVHTRGLDFACDFWLPIGCAAFVVVCILGCILICWLTKKKYSSSVHDPNGEYMFMRAVNTAKKSRLTDVTLRRDQRLPPDAHKPPGGGSFRTPIQEEQADAHSTLAKIKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 16 - protein, 800 aa, synthetic construct
MALPVTALLLPLALLLHAARPQVQLQQSGPGLVKPSQTLSLTCAISGDSVSSNSAAWNWIRQSPSRGLEWLGRTYYRSKWYNDYAVSVKSRITINPDTSKNQFSLQLNSVTPEDTAVYYCAREVTGDLEDAFDIWGQGTMVTVSSGGGGSGGGGSGGGGSDIQMTQSPSSLSASVGDRVTITCRASQTIWSYLNWYQQRPGKAPNLLIYAASSLQSGVPSRFSGRGSGTDFTLTISSLQAEDFATYYCQQSYSIPQTFGQGTKLEIKRTGGGGSGGGGSGGGGSGGGGSGGGGSDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLYCRSKRSRLLHSDYMNMTPRRPGPTRKHYQPYAPPRDFAAYRSKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 17 - protein, 784 aa, synthetic construct
MALPVTALLLPLALLLHAARPEVQLQQSGAELVKPGASVKMSCKASGYTFFSYNMHWVKQTPGQGLEWIGAIYPGNGDTSYNQKFKGKATLTADKSSSTAYMQLSSLTSEDSADYYCARSNYYGSSYWFFDVWGAGTTVTVSSGGGGSGGGGSGGGGSDIVLTQSPAILSASPGEKVTMTCRASSSVNYMDWYQKKPGSSPKPWIYATSNLASGVPARFSGSGSGTSYSLTISRVEAEDAATYYCQQWSFNPPTFGGGTKLEIKGGGGSGGGGSGGGGSGGGGSGGGGSDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACRPAAGGAVHTRGLDFACDFWLPIGCAAFVVVCILGCILICWLTKKKYSSSVHDPNGEYMFMRAVNTAKKSRLTDVTLRRDQRLPPDAHKPPGGGSFRTPIQEEQADAHSTLAKIRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 18 - protein, 789 aa, synthetic construct
MALPVTALLLPLALLLHAARPQVQLQQSGPGLVKPSQTLSLTCAISGDSVSSNSAAWNWIRQSPSRGLEWLGRTYYRSKWYNDYAVSVKSRITINPDTSKNQFSLQLNSVTPEDTAVYYCAREVTGDLEDAFDIWGQGTMVTVSSGGGGSGGGGSGGGGSDIQMTQSPSSLSASVGDRVTITCRASQTIWSYLNWYQQRPGKAPNLLIYAASSLQSGVPSRFSGRGSGTDFTLTISSLQAEDFATYYCQQSYSIPQTFGQGTKLEIKRTGGGGSGGGGSGGGGSGGGGSGGGGSDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACRPAAGGAVHTRGLDFACDFWLPIGCAAFVVVCILGCILICWLTKKKYSSSVHDPNGEYMFMRAVNTAKKSRLTDVTLRRDQRLPPDAHKPPGGGSFRTPIQEEQADAHSTLAKIRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 19 - protein, 789 aa, synthetic construct
MALPVTALLLPLALLLHAARPEVQLQQSGAELVKPGASVKMSCKASGYTFFSYNMHWVKQTPGQGLEWIGAIYPGNGDTSYNQKFKGKATLTADKSSSTAYMQLSSLTSEDSADYYCARSNYYGSSYWFFDVWGAGTTVTVSSGGGGSGGGGSGGGGSDIVLTQSPAILSASPGEKVTMTCRASSSVNYMDWYQKKPGSSPKPWIYATSNLASGVPARFSGSGSGTSYSLTISRVEAEDAATYYCQQWSFNPPTFGGGTKLEIKGGGGSGGGGSGGGGSGGGGSGGGGSDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACRPAAGGAVHTRGLDFACDFWLPIGCAAFVVVCILGCILICWLTKKKYSSSVHDPNGEYMFMRAVNTAKKSRLTDVTLKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 20 - protein, 794 aa, synthetic construct
MALPVTALLLPLALLLHAARPQVQLQQSGPGLVKPSQTLSLTCAISGDSVSSNSAAWNWIRQSPSRGLEWLGRTYYRSKWYNDYAVSVKSRITINPDTSKNQFSLQLNSVTPEDTAVYYCAREVTGDLEDAFDIWGQGTMVTVSSGGGGSGGGGSGGGGSDIQMTQSPSSLSASVGDRVTITCRASQTIWSYLNWYQQRPGKAPNLLIYAASSLQSGVPSRFSGRGSGTDFTLTISSLQAEDFATYYCQQSYSIPQTFGQGTKLEIKRTGGGGSGGGGSGGGGSGGGGSGGGGSDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACRPAAGGAVHTRGLDFACDFWLPIGCAAFVVVCILGCILICWLTKKKYSSSVHDPNGEYMFMRAVNTAKKSRLTDVTLKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 21 - protein, 242 aa, synthetic construct
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 22 - protein, 264 aa, synthetic construct
MALPVTALLLPLALLLHAARPEVQLQQSGAELVKPGASVKMSCKASGYTFFSYNMHWVKQTPGQGLEWIGAIYPGNGDTSYNQKFKGKATLTADKSSSTAYMQLSSLTSEDSADYYCARSNYYGSSYWFFDVWGAGTTVTVSSGGGGSGGGGSGGGGSDIVLTQSPAILSASPGEKVTMTCRASSSVNYMDWYQKKPGSSPKPWIYATSNLASGVPARFSGSGSGTSYSLTISRVEAEDAATYYCQQWSFNPPTFGGGTKLEIK

SEQ ID NO: 23 - protein, 269 aa, synthetic construct
MALPVTALLLPLALLLHAARPQVQLQQSGPGLVKPSQTLSLTCAISGDSVSSNSAAWNWIRQSPSRGLEWLGRTYYRSKWYNDYAVSVKSRITINPDTSKNQFSLQLNSVTPEDTAVYYCAREVTGDLEDAFDIWGQGTMVTVSSGGGGSGGGGSGGGGSDIQMTQSPSSLSASVGDRVTITCRASQTIWSYLNWYQQRPGKAPNLLIYAASSLQSGVPSRFSGRGSGTDFTLTISSLQAEDFATYYCQQSYSIPQTFGQGTKLEIKRT

SEQ ID NO: 24 - protein, 25 aa, synthetic construct
GGGGSGGGGSGGGGSGGGGSGGGGS

SEQ ID NO: 25 - protein, 45 aa, synthetic construct
TTTPAPRPPTPAPTIASQPLSLRPEACRPAAGGAVHTRGLDFACD

SEQ ID NO: 26 - protein, 24 aa, synthetic construct
IYIWAPLAGTCGVLLLSLVITLYC

SEQ ID NO: 27 - protein, 21 aa, synthetic construct
FWLPIGCAAFVVVCILGCILI

SEQ ID NO: 28 - protein, 38 aa, synthetic construct
CWLTKKKYSSSVHDPNGEYMFMRAVNTAKKSRLTDVTL

SEQ ID NO: 29 - protein, 37 aa, synthetic construct
RRDQRLPPDAHKPPGGGSFRTPIQEEQADAHSTLAKI

SEQ ID NO: 30 - protein, 42 aa, synthetic construct
KRGRKKLLYIFKQPFMRPVQTTQEEDGCSCRFPEEEEGGCEL

SEQ ID NO: 31 - protein, 41 aa, synthetic construct
RSKRSRLLHSDYMNMTPRRPGPTRKHYQPYAPPRDFAAYRS

SEQ ID NO: 32 - protein, 112 aa, synthetic construct
RVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR